(19) United States
(12) Patent Application Publication
(10) Pub. No.: US 2007/0110716 A1
(43) Pub. Date: May 17, 2007

(54) Relationship of a specific metabolite to insulin resistance

(76) Inventors: Christopher B. Newgard, Chapel Hill, NC (US); Jie An, Pittsboro, NC (US); Deborah M. Muolo, Chapel Hill, NC (US); Timothy R. Koves, Raleigh, NC (US); David S. Millington, Chapel Hill, NC (US)

Correspondence Address: MYERS BIGEL SIBLEY & SAJOVEC, PO BOX 37428, RALEIGH, NC, 27627, US

(21) Appl. No.: 10/573,207

(22) PCT Filed: Sep. 23, 2004
(86) PCT No.: PCT/US04/31284

§ 371 Date: Jun. 7, 2006

Related U.S. Application Data
(60) Provisional application No. 60/506,601, filed on Sep. 25, 2003.

(51) Int. Cl. A61K 48/00; C12Q 1/68; G01N 33/567; C07H 21/02; A61K 39/395

(52) U.S. Cl. 424/93.2; 424/145.1; 424/93.21; 514/44; 435/456; 536/23.1; 435/7.2; 435/6

(57) ABSTRACT

Provided are methods and reagents for reducing ketone levels (for example, β-hydroxybutyrate) in skeletal muscle. Also provided are methods and reagents for treating insulin resistant states, such as diabetes. Further provided are screening methods for identifying compounds to reduce skeletal muscle ketone levels and/or to treat insulin resistance, for example, insulin resistance associated with diabetes.

### RELATED APPLICATION INFORMATION

This application claims the benefit of priority from U.S. provisional patent application Serial No. 60/506,601, filed Sep. 25, 2003, which is incorporated herein by reference in its entirety.

### STATEMENT OF FEDERAL SUPPORT

The present invention was made, in part, with the support of grant number 5PO1-DK-58398-03 from the National Institutes of Health. The United States government has certain rights to this invention.

### FIELD OF THE INVENTION

The present invention relates to the finding that ketone concentrations in skeletal muscle are related to skeletal muscle and whole animal insulin resistance; in particular, the present invention relates to new therapeutic targets and approaches for the treatment of insulin resistance and diabetes mellitus.

### BACKGROUND OF THE INVENTION

Over one-third of Americans are obese and at high risk for developing type 2 diabetes mellitus, a disease that now affects approximately 100 million people worldwide and whose prevalence is expected to double in the next ten years (Seidell, (2000) Br. J. Nutr. 83(Suppl. 1):S5-S8). Type 2 diabetes is a complex disease that is characterized by disordered energy metabolism and insulin resistance, including the inability of peripheral tissues to respond efficiently to insulin. Skeletal muscle is a major target tissue contributing to whole-body insulin sensitivity. Several lines of evidence link the development of muscle insulin resistance to fatty acid surplus, which often results in inappropriate overstorage of triacylglycerides in muscle tissue (Shulman, (2000) J. Clin Invest. 106:171-76; Schmitz-Peiffer, (2000) Cell Signal 12:583-94; Jucker et al., (1997) J. Biol. Chem. 272:10464473; Krssak et al., (1999) Diabetologia 42:113-16. Various pharmacological and genetic manipulations have been used to show a relationship between depletion of muscle triacylglycerides and concomitant restoration of insulin sensitivity (Gavrilova et al., (2000) J. Clin. Invest. 105:271-78; Kim et al., (2000) J. Biol. Chem. 275:8456-60; Schmitz-Peiffer et al., (1997) Am. J. Physiol. 273:E915-E921; Ye et al., (2001) Diabetes 50:411-17; Zierath et al., (1998) Endocrinology 139:503441; O'Doherty et al., (1999) Am. J. Physiol. 277 (3 Pt1): E544-50). Although triacylcerides alone are thought to be inert lipid storage depots (Goodpaster et al., (2002) Curr Diab. Rep. 2:216-22), abnormally high tissue triacylglyceride levels are proposed to provide excessive substrate for the synthesis of bioactive lipid metabolites that disrupt cell function. A more thorough understanding of how lipid oversupply causes insulin resistance and the precise lipid species that are involved in mediating the pathophysiology is needed for the development of new antidiabetic therapies. Currently, candidate lipid-derived mediators of insulin resistance include long-chain acyl-CoAs, diacylglyerol and ceramide (see Hulver et al., (2003) Am. J. Physiol. Endocrinol. Metab. 284:E741-E747; Cooney et al., (2002) Ann. N.Y. Acad. Sci 967:196-207; Yu et al., (2002) J. Biol. Chem. 277:50230-236), (Yu et al., (2002) J. Biol. Chem. 277:50230-236; Chavez et al., (2003) J. Biol. Chem. 278:10297-303; Hajduch et al., (2001) Diabetologia 44:173-83; Schmitz-Peiffer et al., (1999) J. Biol. Chem. 274:24202-210), (Yu et al., (2002) J. Biol. Chem. 277:50230-236; and Itani et al., (2002) Diabetologia 51:2005-11). However, definitive proof of a cause/effect relationship between the accumulation of these metabolites and insulin resistance is not available.

Ketone bodies, a term that refers to acetoacetate and β-hydroxybutyrate (βHB), the two main ketones, and acetone, which is less abundant, play a key role in sparing glucose and reducing proteolysis during periods of glucose deficiency. The liver is considered the primary site of ketone production. Elevated serum levels of acetoacetate and βHB are strongly associated with insulin resistance in various physiological and pathophysiological energy-stressed states (reviewed in Mitchell et al., (1995) Clin. Invest. Med. 18:193-216), such as starvation (Fujiwara et al., (1988) Diabetes 37:1549-58; Goschke et al., (1977) Metabolism 26:1147-53; Krentz et al., (1992) Diabetes Res. 20:51-60; Mansell et al., (1990) Metabolism 39:502-10), prolonged exercise (Shimomura et al., (1990) J. Appl. Physiol. 68:161-65; Koeslag et al., (1980) J. Physiol. 301:79-90), obesity and type 2 diabetes (Fujiwara et al., (1988) Diabetes 37:1549-58; Goschke et al., (1977) Metabolism 26:1147-53; Krentz et al., (1992) Diabetes Res. 20:51-60; Mansell et al., (1990) Metabolism 39:502-10; Suzuki et al., (1991) Diabetes Res. 18:11-17), severe injury (Williamson, (1981) Acta Chir. Scand. Suppl. 507:22-29; Smith et al., (1975) Lancet 1:1-3), high fat diets (Dell et al., (2001) Lipids 36:373-378) and late-stage pregnancy (Paterson et al., (1967) Lancet 1:862-65; Moore et al., (1989) Teratology 40:237-51). Additionally, antidiabetic drugs, such as tolbutamide (Mori et al., (1992) Metabolism 41:706-10), glitazones (Suzuki et al., (2002) Clin Exp. Pharmacol. Physiol. 29:269-74) and thiazolidinediones (Oakes et al., (1994) Diabetes 43:1203-10) lower circulating ketones. Despite this documented association between elevated circulating ketones and glucose intolerance, the possibility that these metabolites might play a direct role in mediating insulin desensitization has : not been considered. Further, it has not been suggested that abnormal ketogenesis by the skeletal muscle results in this tissue becoming resistant to insulin.

### SUMMARY OF THE INVENTION

The inventors have discovered that skeletal muscle ketone dysregulation is implicated as a novel mechanism linking fatty acid oversupply to insulin resistance. First, mass spectroscopy-based metabolic profiling of skeletal muscle samples from rats in various metabolic states identified a specific lipid-derived intermediate that changes in association with insulin resistance. Thus, animals subjected to fasting or chronic feeding of a high fat (HF) diet (both of which induce insulin resistance) exhibited marked intramuscular accumulation of the ketone, β-hydroxybutyrate (βHB). Second, adenovirus-mediated delivery of a lipid catabolic enzyme, malonyl-CoA decarboxylase (MCD), to liver resulted in the near complete reversal of muscle insulin resistance caused by HF feeding and also caused a 55% decrease in muscle βHB levels, with little or no change in other lipid intermediates. Moreover, these changes in intramyocellular βHB were likely due to changes in the metabolism of the ketone within muscle tissue, as no significant change in βHB levels occurred in plasma or in liver of HF fed animals in response to hepatic MCD expression. The discovery of the connection between accumulation of ketones in skeletal muscle and insulin resistance opens up the possibility of new therapeutic approaches for treating insulin resistance and, in particular, diabetes.

Accordingly, the invention provides a method of treating diabetes by reducing the accumulation of ketones in skeletal muscle. As one aspect, the invention provides a method of treating diabetes comprising administering a compound that reduces skeletal muscle ketone levels to a diabetic subject in a therapeutically effective amount to reduce skeletal muscle ketone levels.

As another aspect the invention provides a delivery vector comprising a heterologous nucleic acid that encodes a ketolytic enzyme operably linked to a control element that directs the expression of the nucleic acid in skeletal muscle cells.

As a further aspect, the invention provides a delivery vector comprising a heterologous nucleic acid that encodes an enzyme that mediates fatty acid oxidation operably linked to a control element that directs the expression of the nucleic acid in hepatic cells.

Also provided by the invention is an inhibitory oligonucleotide (e.g., that is at least 8 nucleotides in length) that specifically hybridizes to a target sequence encoding a ketogenic enzyme and reduces production of the ketogenic enzyme. In particular embodiments, the inhibitory oligonucleotide is an antisense molecule or an RNAi molecule. The invention also provides a delivery vector comprising a heterologous nucleic acid encoding the inhibitory oligonucleotide, optionally linked to a control element that directs the expression of the nucleic acid in skeletal muscle cells.

As another aspect, the invention provides pharmaceutical formulations comprising the delivery vectors and inhibitory oligonucleotides described herein.

As still a further aspect, the invention provides methods of reducing ketone levels in skeletal muscle using the delivery vectors, inhibitory oligonucleotides, and pharmaceutical formulations set forth herein. The methods can be carried out in vitro or in vivo.

As yet another aspect, the invention provides methods of treating insulin resistance and diabetes using the delivery vectors, inhibitory oligonucleotides, and pharmaceutical formulations set forth herein.

As still other aspects, the invention provides cell-free, cell-based and whole animal methods of identifying a candidate compound for reducing skeletal muscle ketone levels, treating insulin resistance and/or treating diabetes.

These and other aspects of the invention are set forth in more detail in the description of the invention below.

### BRIEF DESCRIPTION OF THE DRAWINGS

FIG. 1. A Proposed Model of Ketone Regulation in Skeletal Muscle. Ketone homeostasis in muscle relies on a balance between the supply of hepatic ketones, production of endogenously synthesized ketones and ketone degradation. Ketones enter peripheral tissues by passive diffusion or via the monocarboxylic family of transporters (MCT). The reversible conversion between βOH-butyrate (βHB) and acetoacetate (AcAc) is catalyzed by βOH-butyrate dehydrogenase (βHBD). AcAc is then converted to acetoacetyl-CoA by succinyl-CoA:3oxoacid CoA transferase (SCOT), which represents the rate-determining step in ketolysis. Energy stress activates branched-chain ketoacid dehydrogenase (BCKAD), the enzyme that catalyzes the rate-limiting step in the conversion of leucine to HMG-CoA. Leucine is the main ketogenic amino acid and under some conditions becomes a major energy-providing substrate for skeletal muscle. De novo synthesis of HMG-CoA requires HMG-CoA synthase (mHS), a mitochondrial enzyme that is expressed most abundantly in liver but has also been detected in skeletal muscle. mHS catalyzes the condensation of acetyl-CoA with AcAc-CoA, which is the product of the 3-ketothiolase (3-KT) reaction. HMG-CoA is cleaved by HMG-CoA lyase (HL) to produce AcAc and acetyl-CoA. These products can be oxidized as energy substrates or converted to βHB. Several of the key regulatory steps in ketogenesis are induced ⊕ by high fatty acid (FA) and/or peroxisome proliferator receptor (PPAR) agonists. Conversely, FA and PPAR agonists inhibit Θ the pyruvate dehydrogenase (PDH) reaction, thereby favoring anaplerotic entry of pyruvate into the tricarboxylic acid (TCA) cycle via pyruvate carboxylase (PC) or the malic enzyme (ME). Anaplerotic flux of carbons into the TCA is enhanced during metabolic states in which ketones become a dominant energy substrate. Studies in isolated heart suggest that ketones inhibit the α-ketoacid dehydrogenase (αKAD) reaction, thereby diminishing cellular levels of succinyl-CoA. Under these circumstances, TCA cycle flux is maintained only upon provision of anaplerotic substrates, such as pyruvate or lactate. Since succinyl-CoA is a key negative regulator of mHS, ketone-induced suppression of αKAD may serve as a feed forward signal that further promotes ketogenesis.

FIG. 2 is a model illustrating the unique role for succinyl-CoA in regulating muscle ketone homeostasis as suggested by its involvement in three independent enzymatic reactions that cooperatively favor βHB catabolism over synthesis. First, succinyl-CoA functions as a potent negative regulator of the ketogenic enzyme, mHS. Studies in rat liver have shown that succinyl-CoA inhibits mHS through both an allosteric mechanism and via a covalent reaction that results in enzyme succinylation and inactivation. Succinyl-CoA-mediated inhibition of mHS plays an important physiological role in suppressing hepatic ketogenesis during the starved to fed transition and in response to high carbohydrate feeding. Second, succinyl-CoA reacts with the ketolytic enzyme, SCOT, in converting AcAc to AcAc—CoA. Thus, high succinyl-CoA levels favor diversion of AcAc towards oxidation and away from the βHBD reaction. Finally, because succinyl-CoA also functions as a TCA cycle intermediate, its depletion can impede oxidative flux and force accumulation of acetyl-CoA. High ketone levels have been shown to lower succinyl-CoA levels by inhibiting its production via 60 ketoglutarate dehydrogenase complex (αKGD). This model therefore predicts that raising intramuscular succinyl-CoA levels would oppose βHB accumulation and promote insulin sensitivity.

FIG. 3 shows exemplary target sequences from ketogenic enzymes for the design of RNAi.

FIG. 4 shows MCD activity and palmitate oxidation in primary hepatocytes. Hepatocytes were isolated from fed rats and treated with recombinant adenoviruses containing a catalytically inactive form of MCD (AdCMV-MCDₘᵤₜ), a catalytically active form that is preferentially localized to the cytosol (AdCMV-MCDΔ5), or left untreated. The construction of the recombinant adenoviruses used is described in detail in Mulder et al., (2001) J. Biol. Chem. 276:6479-84. Cell extracts were prepared from parallel cultures for measurement of MCD enzymatic activity or palmitate oxidation 48 hours after viral. treatment. FIG. 3A shows MCD activity. FIG. 3B shows ³H palmitate oxidation. Data represent the mean±S.E. of four independent experiments, and the symbol * indicates differences between AdCMV-MCDΔ5-treated cells and the two control groups, with p≦0.001.

FIG. 5 shows evidence for restoration of muscle insulin signaling by hepatic expression of MCD in HF rats. Normal Wistar rats were fed on standard chow (SC) or high-fat diet (HF) for 11 weeks prior to injection of AdCMV-MCDₘᵤₜ or AdCMV-MCDΔ5 as indicated. Muscle samples were prepared, resolved by SDS-PAGE, and immunoblotted with antibodies specific for phosph-AKT-1 (Ser⁴⁷³), AKT-2, phospho-GSK-30(Ser⁹) and total AKT. Data are shown for duplicate samples for each experimental group and are representative of two similar experiments.

FIG. 6 shows liver and muscle triglyceride levels. Normal Wistar rats were fed on a standard chow (SC) or high-fat diet (HF) for 11 weeks prior to injection of AdCMV-MCDₘᵤₜ (white bars) or AdCMV-MCDΔ5 (shaded bars) and tissue triglyceride (TG) levels were measured as described herein. Panel A, Liver TG in rats fed on the SC or HF diet. Panel B. Muscle TG in gastrocnemius, soleus and extensor digitorum longus (EDL) from rats fed on the HF diet. Data represent the mean±S.E. of 8 to 13 animals for liver and gastrocnemius muscle and 4 animals for soleus and EDL muscles. The symbol * in Panel A indicates that liver TG was lower in AdCMV-MCDΔ5-treated compared to AdCMV-MCDₘᵤₜ-treated HF rats, with p≦0.001. The symbol * in Panel B indicates that gastrocnemius muscle TG was higher in AdCMV-MCDΔ5-treated compared to AdCMV-MCDₘᵤₜ-treated HF rats, with p≦0.05.

FIG. 7 shows that βOH-butyrate (βHB) carnitine esters in muscle increase with starvation and high fat diet. Gastrocnemius muscles were harvested from rats starved or fed rats after 10 weeks on a high-fat (HF) or standard chow (SC) diet. Acylcarnitine levels were analyzed by tandem MS/MS. * C5=carnitine ester of isovaleryl-CoA, an intermediate in leucine degradation. * C4-OH=carnitine ester of βOHbutyrate.

FIG. 8 shows that elevated βHB carnitine esters in muscles from HF fed rats are restored to normal levels by MCD treatment. Gastrocnemius muscles were harvested from starved rats fed on a high fat (HF) diet for 8 weeks. Acylcarnitine levels were analyzed by tandem MS/MS. * C4-OH=carnitine ester of βOHbutyrate.

FIG. 9 shows a summary of βOH results from two experiments. βOH values were pooled from two independent experiments, shown in FIG. 7 and FIG. 8.

FIG. 10 shows that a high fat diet does not increase βHB in liver. Panel A shows the levels of acylcarnitine in the liver of animals starved or fed a high-fat (HF) or standard chow (SC) diet. Panel B shows the levels of acylcarnitine in the liver of animals fed a high fat diet and receiving MCD treatment. C4-OH=carnitine ester of βOHbutyrate.

FIG. 11 shows mHS expression in Rat L6 myotubes. Myocytes were incubated in standard media (control) or with 500 μM oleate (FA) for 24 hours. Total RNA was isolated by the TriZol method and gene expression levels were quantified by RTQ-PCR. Shown are representative samples that were also analyzed by standard RT-PCR. mHS, mitochondrial HMG-CoA synthase. G6PDH, glucose 6 phosphate dehydrogenase. Data are representative of three independent experiments. These data demonstrate that mHS, an enzyme normally considered to be primarily expressed in liver, also is present in muscle and is upregulated when lipids are abundant, as occurs in type 2 diabetes.

FIG. 12 shows acylcarnitine levels in gastrocnemius muscles of rats fed a standard chow (SC) or high fat (HF) diet.

FIG. 13 shows oleate oxidation (Panel A) in mitochondria isolated from gastrocnemius muscles of rats fed a standard show (SC) or high fat (HF) diet (Panel B), or treated with streptozotocin (STZ) (Panel C).

FIG. 14, panels A-F, shows acylcarnitine accumulation in L6 myocytes incubated 24 h in differentiation medium without FA (DFM) or with 500-1000 μM 2:1 oleate/palmitate (O/P) or palmitate oleate (P/O). Panel G, the ratio of complete (CO₂) to incomplete (ASM) [¹⁴C]oleate oxidation decreases as FA supply increases.

FIG. 15. Insulin-stimulated phosphorylation of Akt. High palmitate-(Panel A) and high oleate-(Panel B) induced insulin resistance requires carnitine. NT; no FA treatment. Panel C, Etomoxir attenuates lipid-induced insulin resistance.

### DETAILED DESCRIPTION OF THE INVENTION

The present invention is based in part on new insights gained from application of mass spectroscopy-based metabolic profiling to skeletal muscle samples from normally insulin sensitive and insulin resistant animals. These findings define a heretofore undescribed correlation between the concentration of ketones within the muscle tissue (e.g., β-hydroxybutyrate; βHB) and insulin sensitivity. This relationship is seen in three independent experimental models: (1) comparison of animals fed on a high fat (HF) diet and animals fed on normal chow (the former are insulin resistant); (2) fasted versus fed animals (the former are insulin resistant); and (3) animals fed on a high fat diet that are engineered for expression of an enzyme in liver that alters lipid partitioning. Expression of this enzyme, malonyl CoA decarboxylase (MCD), in the liver of animals fed on a HF diet results in reversal of insulin resistance in skeletal muscle. Animals with MCD expression that have normalized insulin sensitivity experience a profound decrease in levels of βHB concentrations in muscle that correlates with the return of insulin sensitivity. Moreover, the fall in muscle βHB concentration occurs independent of changes in other, structurally related lipid-derived intermediates. Further, the striking fall in muscle ketone levels seen in response to MCD expression in liver of HF-fed rats are not paralleled in liver extracts from the same animals, nor are significant changes detected in circulating ketone levels. These data indicate that the changes in ketone levels in muscle that relate to changes in insulin sensitivity are mediated by changes in intramuscular synthesis and/or degradation of ketones, rather than changes in hepatic production and delivery to muscle.

The novel finding that ketone concentrations in skeletal muscle correlate with whole animal and skeletal muscle insulin resistance provides new possibilities for therapeutic interventions in insulin resistant states, such as type 2 diabetes, in which the prevention and/or reversal of ketone accumulation in skeletal muscle are targeted. The invention therefore provides new therapeutic approaches and targets for the treatment of insulin resistant states.

The present invention will now be described with reference to the accompanying drawings, in which preferred embodiments of the invention are shown. This invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference in their entirety.

Except as otherwise indicated, standard methods can be used for the production of viral and non-viral vectors, manipulation of nucleic acid sequences, production of transformed cells, and the like according to the present invention. Such techniques are known to those skilled in the art. See, e.g., SAMBROOK et al., MOLECULAR CLONING: A LABORATORY MANUAL 2nd Ed. (Cold Spring Harbor, N.Y., 1989); F. M. AUSUBEL et al. CURRENT PROTOCOLS IN MOLECULAR BIOLOGY (Green Publishing Associates, Inc. and John Wiley & Sons, Inc., New York).

I. Definitions.

As used in the description of the invention and the appended claims, the singular forms “a,” “an” and “the” are intended to include the plural forms as well, unless the context clearly indicates otherwise.

As used herein, the term “diabetes” is used interchangeably with the term “diabetes mellitus.” The terms “diabetes” and “diabetes mellitus” are intended to encompass both insulin dependent and non-insulin dependent (Type I and Type II, respectively) diabetes mellitus, unless one condition or the other is specifically indicated.

By “insulin resistance” or “insulin insensitivity” it is meant a state in which a given level of insulin produces a less than normal biological effect (e.g., uptake of glucose). Insulin resistance is particularly prevalent in obese individuals or those with type 2 diabetes. In type 2 diabetics, the pancreas is generally able to produce insulin, but there is an impairment in insulin action. As a result, hyperinsulinemia is commonly observed in insulin resistant subjects. Insulin resistance is less common in type I diabetics; although in some subjects, higher dosages of insulin have to be administered over time indicating the development of insulin resistance/insensitivity. The term “insulin resistance” or “insulin insensitivity” refers to whole animal insulin resistance/insensitivity unless specifically indicated otherwise. Methods of evaluating insulin resistancelinsensitivity are known in the art, for example, hyperinsulinic/euglycemic clamp studies, insulin tolerance tests, uptake of labeled glucose and/or incorporation into glycogen in response to insulin stimulation, and measurement of known components of the insulin signalling pathway (e.g., phosphorylation of Akt proteins).

One standard methodology for the evaluation of insulin resistance is the hyperinsulinemic/euglycemic clamp. An exemplary protocol is as follows: catheters are placed at least two weeks in advance into the ileal vein, common carotid artery, and right external jugular vein in laboratory rats under general anesthesia (e.g., pentobarbital sodium; 50 mg/kg, ip). Experiments are performed on overnight-fasted conscious animals that are allowed to move freely. Each experiment consists of a 90-minute tracer equilibration period (−150 to −60 minutes), a 60-minute control period (−60 to 0 minutes), and a 180-minute clamp period (0 to 180 minutes). The tracers are infused through the jugular vein catheter. A priming dose of [3-³H] glucose (10 μCi) and [U-¹⁴C] glucose (10 μCi) is given at −150 minutes. Continuous infusions of [3-³H], [U-¹⁴C] glucose are also started at −150 minutes. During the clamp period, somatostatin is infused through the jugular catheter continuously at 2 μg·kg⁻¹ min⁻¹ to inhibit endogenous insulin and glucagon production. Glucagon and insulin are infused through the ileal vein catheters to maintain plasma glucagon and insulin levels at ˜30 pg/mL and ˜3 ng/mL, respectively. Blood glucose is monitored every 10 minutes via. carotid arterial catheter samples. Glucose is infused through the jugular catheter as required to maintain euglycemia.

An “improvement in insulin resistance” is a level of improvement that provides some clinical benefit to the subject. Insulin resistance can be assessed as described in the preceding paragraph. In particular embodiments, an “improvement in insulin resistance” can result in normalization of insulin sensitivity.

A “transgenic” non-human animal is a non-human animal that comprises a foreign nucleic acid incorporated into the genetic makeup of the animal such as, for example, by stable integration into the genome or by stable maintenance of an episome (e.g., derived from EBV).

A “therapeutically effective” amount as used herein is an amount that provides some improvement or benefit to the subject. Alternatively stated, a “therapeutically effective” amount is an amount that provides some alleviation, mitigation and/or decrease in at least one clinical symptom of insulin resistance or diabetes in the subject (e.g., improved glucose tolerance, enhanced insulin-stimulated glucose uptake, improved serum insulin concentrations, and the like) as is well-known in the art. Those skilled in the art will appreciate that the therapeutic effects need not be complete or curative, as long as some benefit is provided to the subject.

The terms “treat,” “treating” or “treatment of” (or grammatically equivalent terms) it is meant that the severity of the patient's condition is reduced or at least partially improved or ameliorated and/or that some alleviation, mitigation or decrease in at least one clinical symptom is achieved and/or there is a delay in the progression of the condition and/or prevention or delay of the onset of a disease or illness.

As used herein, a “delivery vector” can be a viral or non-viral (e.g., lipid based) vector that is used to deliver a nucleic acid to a cell, tissue or subject.

A “recombinant” vector or delivery vector refers to a viral or non-viral vector that comprises one or more heterologous nucleic acids, e.g., two, three, four, five or more heterologous nucleic acids.

A “heterologous nucleic acid” will typically be a sequence that is not naturally-occurring in the vector. Alternatively, a heterologous nucleic acid can refer to a sequence that is placed into a non-naturally occurring environment (e.g., by association with a promoter with which it is not naturally associated).

As used herein, the term “polypeptide” encompasses both peptides and proteins, unless indicated otherwise.

A “recombinant” nucleic acid is one that has been created using genetic engineering techniques.

A “recombinant polypeptide” is one that is produced from a recombinant nucleic acid.

As used herein, an “isolated” nucleic acid (e.g., an “isolated DNA” or an “isolated vector genome”) means a nucleic acid separated or substantially free from at least some of the other components of the naturally occurring organism or virus, such as for example, the cell or viral structural components or other polypeptides or nucleic acids commonly found associated with the nucleic acid. Isolated nucleic acids of this invention include RNA, DNA (including cDNAs) and chimeras thereof. The isolated nucleic acids can further comprise modified nucleotides or nucleotide analogs.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide. As used herein, the “isolated” polypeptide can be at least about 25%, 50%, 60%, 70%, 75%, 80%, 85%, 90%, 95%, 97%, 98%, 99% or more pure (w/w).

By the term “express” or “expression” (and grammatical equivalents thereof) of a nucleic acid coding sequence, it is meant that the sequence is transcribed, and optionally, translated.

By “skeletal muscle cell” it is meant a cultured cell, a cell in a tissue culture or explant, or a cell in vivo. Cultured muscle cells include primary myoblast or myotube cultures as well as immortalized myogenic cell lines such as the L6 and C6C12 cell lines.

By “liver cell” it is meant a cultured cell, a cell in a tissue or organ culture, or a cell in vivo. Cultured liver cells include primary hepatocyte cultures as well as immortalized cell lines such hepatoma cell lines. Typically, the term “liver cell” refers to a parenchymal cell.

II. Regulating Ketone Concentrations in Skeletal Muscle.

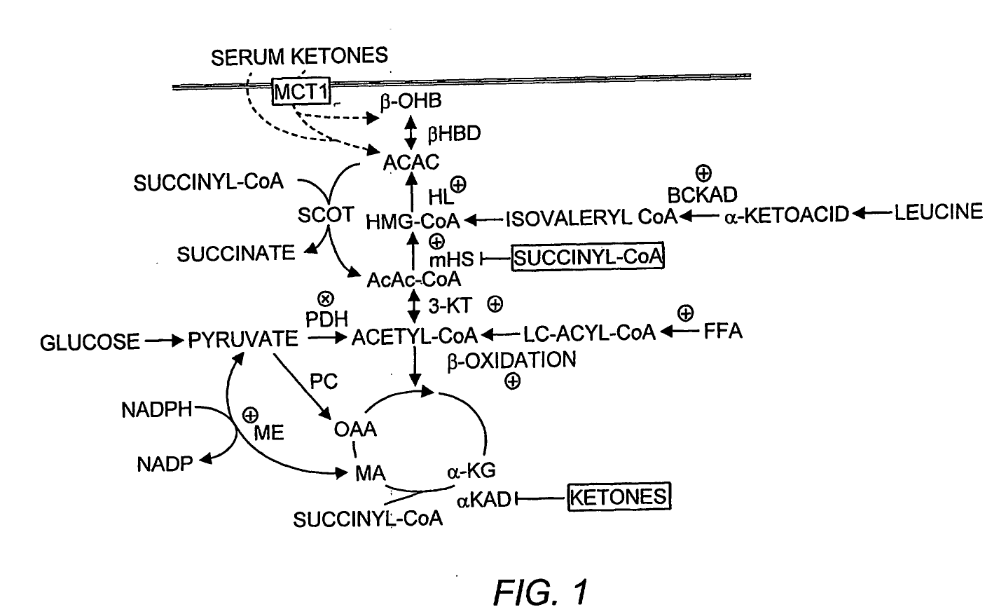

FIG. 1 illustrates a model of ketone metabolism in skeletal muscle and highlights some of the key regulatory pathways. Ketone homeostasis relies on a balance between the supply of hepatic ketones, production of endogenously synthesized ketones and ketolysis. Ketones can enter peripheral tissues by passive diffusion or via the monocarboxylic acid family of transporters (MCT). The reversible conversion between βHB and acetoacetate (AcAc) is catalyzed by βOH-butyrate dehydrogenase (βHBD). AcAc is then converted to acetoacetyl-CoA by the enzyme succinyl-CoA:3oxoacid CoA transferase (SCOT), which is abundant in muscle and considered the rate-determining enzyme in ketolysis.

Ketogenesis is a mitochondrial process by which acetyl-CoA, mostly derived from the β-oxidation of fatty acids, is converted to the ketone bodies, AcAc, βHB and acetone. As shown in FIG. 1, this conversion occurs in four reactions catalyzed sequentially by acetoacetyl-CoA thiolase, mitochondrial HMG-CoA synthase (mHS), HMG-CoA lyase (HL) and βHBD. Ketogenesis occurs mainly in liver, but can also occur in other non-hepatic tissues including kidney, brain, heart and skeletal muscle.

The present invention provides reagents and methods for reducing (e.g., by at least about 20%, 25%, 35%, 40%, 50%, 60%, 75%, 85%, 90%, 95% or more) or normalizing ketone levels in skeletal muscle. Also provided are reagents and methods for treating insulin resistance, for example in . diabetes (in particular, type 2 diabetes) by reducing or normalizing ketone levels in skeletal muscle. To illustrate, ketogenesis can be reduced or normalized and/or ketolysis enhanced or normalized in skeletal muscle, liver or any other ketogenic tissue. As another approach, the availability of ketogenic precursors (such as non-esterified free fatty acids) to the skeletal muscle can be reduced, e.g., by enhancing lipid oxidation (i.e., fatty acid oxidation) in the liver.

By “normalizing” enzyme activity, ketone concentrations and the like it is meant that the indicated activity or concentration is altered to the level observed in the absence of insulin resistance (e.g., in a healthy subject).

The term “ketones” or “ketone bodies” generally refers to acetone, acetoacetate and β-hydroxybutyrate (βHB). In particular embodiments, the invention is practiced to specifically reduce and/or to detect βHB.

Those skilled in the art will appreciate that the terms “ketolytic enzyme,” “ketogenic enzyme,” and “lipid oxidizing enzyme” (i.e., an enzyme that mediates free fatty acid oxidation), and the like, as used herein encompass both the full-length enzymes as well as functional portions thereof that retain enzymatic activity (e.g., at least about 50%, 60%, 75%, 85%, 90%, 95%, 97%, 98%, 99%, or more, of the enzymatic activity of the full-length enzyme). Also encompassed are other modified forms (e.g., to change the subcellular localization) of the enzymes, that retain enzymatic activity, as defined above.

In representative embodiments of the invention, the activity of a ketogenic enzyme(s) is reduced (e.g., by at least about 20%, 25%, 35%, 40%, 50%, 60%, 75%, 85%, 90%, 95% or more) or normalized. In other embodiments, the activity of a ketolytic enzyme(s) is enhanced (e.g., by at least about 20%, 25%, 35%, 40%, 50%, 65%, 75%/, 100%, 125%, 150%, 200% or more) or normalized in skeletal muscle and/or liver.

In other illustrative embodiments, the activity of a lipid oxidizing enzyme(s) is enhanced (e.g., by at least about 20%, 25%, 35%, 40%, 50%, 65%, 75%,100%, 125%,150%,200% or more) or normalized in the liver.

The modulation (i.e., reduction or enhancement) of enzyme activity can be a result of a change in enzyme levels and/or a change in the biological activity of the enzyme, and further can be effected at the nucleic acid or protein level.

Thus, as one aspect, the invention provides a method of reducing ketone levels in a skeletal muscle cell comprising contacting a skeletal muscle cell with a delivery vector comprising a heterologous nucleic acid encoding a ketolytic enzyme (optionally, the heterologous nucleic acid is operably linked to a control element that directs the expression of the heterologous nucleic acid in skeletal muscle cells) in an amount effective to reduce ketone levels in the skeletal muscle cell. In particular embodiments, the ketolytic enzyme is acetoacetate:succinyl CoA:3oxoacid CoA transferase (SCOT) and/or α-ketoacid dehydrogenase.

As another embodiment, the invention provides a method of treating insulin resistance or diabetes (in particular, type 2 diabetes) comprising administering a pharmaceutical formulation comprising a delivery vector comprising a heterologous nucleic acid encoding a ketolytic enzyme (optionally, the heterologous nucleic acid is operably linked to a control element that directs the expression of the heterologous nucleic acid in skeletal muscle cells) to the skeletal muscle of an insulin resistant or diabetic subject in a therapeutically effective amount to reduce or even normalize skeletal muscle ketone levels.

SCOT (EC 2.8.3.5), which is also known as 3-oxoacid CoA transferase 1 is a homodimeric mitochondrial matrix enzyme. It is an important enzyme in the extrahepatic utilization of ketones, catalyzing the reversible transfer of coenzyme A from succinyl-CoA to acetoacetate, a necessary step in ketolytic energy production. The nucleic acid and amino acid sequences of various SCOT enzymes are known (see, e.g., Accession No. NM_{—}000436; tissue type: heart, subcellular localization: mitochondrial; Kassovska-Bratinova, et al. (1996) Am. J. Hum. Genet. 59(3):519-528).

α-ketoacid dehydrogenase is a multienzyme complex associated with the inner membrane of mitochondria, and functions in the catabolism of branched-chain amino acids. The complex consists of multiple copies of 3 components: branched-chain α-keto acid decarboxylase (E1), lipoamide acyltransferase (E2) and lipoamide dehydrogenase (E3). The gene, BCKDHB E1-beta, encodes the E1 beta subunit, and mutations therein have been associated with maple syrup urine disease (MSUD), type 1B. Alternative splicing at this locus results in transcript variants with different 3′ noncoding regions, but encoding the same isoform. Transcript variants encoding α-ketoacid dehydrogenase are known (Chuang, et al. (1996) Am. J. Hum. Genet. 58(6):1373-1377). Variant 1 (Accession no. NM_{—}183050) represents the longer transcript. Variant 2 (Accession no. NM_{—}000056) is missing a segment in the 3′ UTR compared with transcript variant 1, and thus has a shorter 3′ UTR. Both variants 1 and 2 encode the same protein.

Alternatively, muscle ketone levels can be lowered by a reduction in the delivery of circulating free fatty acids. In exemplary embodiments, lipid partitioning in the liver is affected by manipulation of malonyl CoA levels (e.g., by overexpressing malonyl CoA decarboxylase in the liver).

Thus, the invention provides methods of reducing ketone levels in a skeletal muscle cell comprising contacting a liver cell with a delivery vector comprising a heterologous nucleic acid encoding a lipid oxidizing enzyme (i.e., a fatty acid oxidizing enzyme) in an amount effective to reduce ketone levels in the skeletal muscle cell. Optionally, the heterologous nucleic acid is operably linked to a control element that directs the expression of the heterologous nucleic acid in liver cells.

The invention further encompasses methods of treating insulin resistance or diabetes (in particular, type 2 diabetes) comprising administering a pharmaceutical formulation comprising a delivery vector comprising a heterologous nucleic acid encoding a lipid oxidizing enzyme (optionally, the heterologous nucleic acid is operably linked to a control element that directs the expression of the heterologous nucleic acid in liver cells) to the liver of an insulin resistant or diabetic subject in a therapeutically effective amount to reduce or even normalize skeletal muscle ketone levels.

In exemplary embodiments, the activity of lipid oxidizing enzymes is increased, including malonyl CoA decarboxylase, carnitinepalmitoyl transferase I, carnitinepalmitoyl transferase II, carnitine acyltranslocase, acyl-CoA dehydrogenase, enoyl-CoA hydratase, 3-L-hydroxyacyl-CoA dehydrogenase and/or β-ketoacyl-CoA thiolase. In other representative embodiments, the enzyme is not malonyl CoA decarboxylase.

Malonyl CoA decarboxylase (EC 4.1.1.9) is encoded by the MLYCD gene and catalyzes the conversion of malonyl-CoA to acetyl-CoA and carbon dioxide. This enzyme exists as peroxisomal, mitochondrial and cytoplasmic forms. The nucleic acid and amino acid sequences of malonyl CoA decarboxylase are known (see, e.g., Accession No. NM_{—}012213 [cytoplasmic and peroxisomal localization], Gao, et al. (1999) J. Lipid Res. 40(1):178-182); Accession No. AF097832 [peroxisomal and mitochondrial localization], Fitzpatrick, et al. (1999) Am. J. Hum. Genet. 65(2):318-326). In particular embodiments, the malonyl CoA is modified so that it is localized to the cytoplasm rather than the mitochondrion or peroxisome (see, e.g., Mulder et al., (2001) J. Biol. Chem. 276:6479-84).

Carnitine palmitoyltransferase I and II (EC 2.3.1.21; CPT-1 and CPT-2) oxidize long-chain fatty acids in the mitochondria. Defects in these proteins are associated with mitochondrial long-chain fatty-acid (LCFA) oxidation disorder. The nucleic acid and amino acid sequences of CPT-1 and CPT-2 are known, see, e.g., Accession No. NM_{—}004377 (CPTIB; human, skeletal muscle); Yamazaki et al. (1996) Biochim. Biophys. Acta 1307(2):157-161); Accession No. NM_{—}001876 (CPT1A; human, liver; Britton et al. (1995) Proc. Natl. Acad. Sci. U.S.A. 92(6):1984-1988); and Accession No. NM_{—}000098 (CPT2; mitochondrial; Finocchiaro, et al. (1991) Proc. Natl. Acad. Sci. U.S.A. 88(2):661-665.

Carnitine acetyltransferase (CRAT; EC 2.3.1.7) is an enzyme in the metabolic pathway in mitochondria, peroxisomes and endoplasmic reticulum. CRAT catalyzes the reversible transfer of acyl groups from an acyl-CoA thioester to carnitine and regulates the ratio of acylCoA/CoA in the subcellular compartments. Different subcellular localizations of the CRAT mRNAs are thought to result from alternative splicing of the CRAT gene suggested by the divergent sequences in the 5′ region of peroxisomal and mitochondrial CRAT cDNAs and the location of an intron where the sequences diverge. The alternative splicing of this gene results in three distinct isoforms, one of which contains an N-terminal mitochondrial transit peptide, and has been shown to be located in mitochondria.

There are a number of known CRAT transcript variants. Transcript Variant 1 is also known as the mitochondrial transcript variant. It encodes the longest isoform 1 that contains a mitochondrial leader peptide. Transcript Variant 2 is known as the peroxisomal transcript variant. It includes a unique 5′ region as compared with variant 1. The translation begins at a downstream in-frame start codon, and results in isoform 2 that contains a shorter N-terminus compared to isoform 1. Transcript Variant 3 lacks a segment in the coding region compared to variant 1. The translation remains in-frame, and results in an isoform 3 that lacks an internal region compared to isoform 1. Various nucleic acid and amino acid sequences of CRAT are known, see, e.g., Accession No. NM_{—}000755 (transcript variant 1; human, mitochondrial; Corti, et al. (1994) Genomics 23(1):94-99); Accession No. NM_{—}004003 (transcript variant 2; human, peroxisomal; Corti, et al. (1994) Genomics 23(1):94-99); and Accession No. NM_{—}144782 (transcript variant 3; human; Corti, et al. (1994) Genomics 23(1):94-99).

Acyl-CoA dehydrogenase (EC 1.3.99.3, EC 1.3.99.12, EC 1.3.99.2 and EC 1.3.99.13) catalyzes the initial step of the mitochondrial fatty acid β-oxidation pathway. The enzyme exists in a variety of forms that are specific for short-, medium-, long- and very long-chain fatty acids. The nucleic acid and amino acid sequences of a variety of acyl-CoA dehydrogenase enzymes are known, see, e.g., Accession No. NM_{—}014384 (ACAD8, Telford et al. (1999) Biochim. Biophys. Acta 1446(3):371-376); Accession No. NM_{—}014049 (ACAD9, Zhang et al. (2002) Biochem. Biophys. Res. Commun. 297(4):1033-1042); Accession No. NM_{—}000016 (ACADM, Kelly et al. (1987) Proc. Natl. Acad. Sci. U.S.A. 84(12):4068-4072); Accession No. NM_{—}000018 (ACADVL, Aoyama, et al. (1995) Am. J. Hum. Genet. 57(2):273-283); Accession No. NM_{—}000017 (ACADS, Naito, et al. (1989) J. Clin. Invest. 83(5):1605-1613); Accession No. NM_{—}001609 (ACADSB; Rozen, et al. (1994) Genomics 24(2):280-287); and Accession No. NM_{—}001608 (ACADL; Indo, et al. (1991) Genomics 11 (3):609-620).

Enoyl-CoA hydratase (EC 4.2.1.1) is encoded by the ECHS1 gene, is localized to the mitochondrial matrix, and functions in the second step of the mitochondrial fatty acid β-oxidation pathway. It catalyzes the hydration of 2-trans-enoyl-coenzyme A (CoA) intermediates to L-3-hydroxyacyl-CoAs. Transcript variants utilizing alternative transcription initiation sites have been described in the literature. For illustrative nucleic acid and amino acid sequences of enoyl-CoA hydratase enzymes, see e.g., Accession No. NM_{—}004092 (Kanazawa, et al. (1993) Enzyme Protein 47(1):9-13).

3-L-hydroxyacyl-CoA dehydrogenase catalyzes the oxidation of 3-L-hydroxylacyl-CoA to β-ketoacyl-CoA+NADH+H⁺ in the third step of the β-oxidation pathway (see, e.g., Accession No. NM_{—}005327; [liver] Vredendaal, et al. (1996) Biochem. Biophys. Res. Commun. 223(3):718-723) and Accession No. AF001903; isoform 2 [skeletal muscle] Samuel and Jung, unpublished).

β-ketoacyl-CoA thiolase (EC 2.3.1.16) is also known as the ACAT2 form of acetyl-CoA acetyltransferase. β-ketoacyl-CoA thiolase catalyzes the conversion of β-ketoacyl-CoA+CoASH to fatty acyl-CoA+acetyl-CoA in the final step of the β-oxidation pathway (Goldman, (1954) J. Biol. Chem. 208:345-57. Nucleic acid and amino acid sequences of β-ketoacyl-CoA thiolase are known in the art (see, e.g., Accession No. NM_{—}6111).

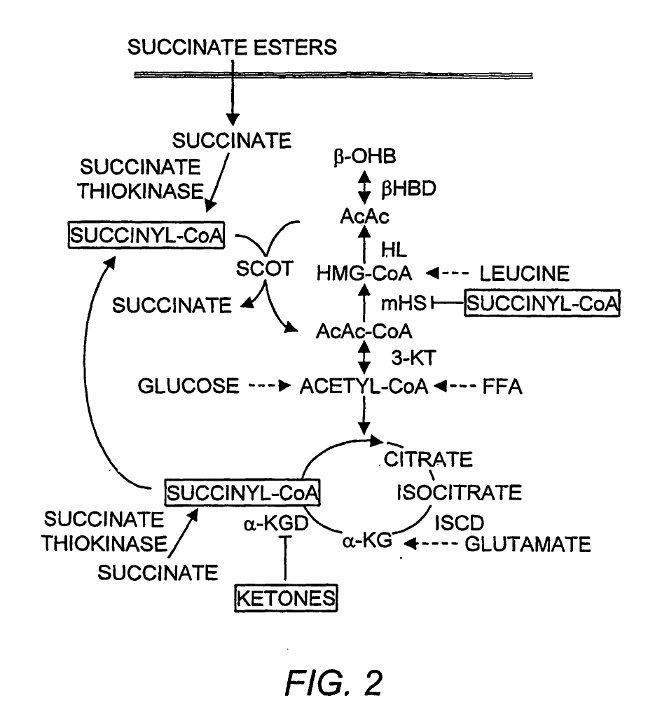

As still another approach, the accumulation of ketones in skeletal muscle can be reduced or normalized by enhancing succinyl CoA levels in skeletal muscle. A unique role for succinyl-CoA in regulating muscle ketone homeostasis is suggested by its involvement in three independent enzymatic reactions that cooperatively favor βHB catabolism over synthesis (FIG. 2). First, succinyl-CoA functions as a potent negative regulator of the ketogenic enzyme, mHS. Studies in rat liver have shown that succinyl-CoA inhibits mHS through both an allosteric mechanism and via a covalent reaction that results in enzyme succinylation and inactivation. Succinyl-CoA-mediated inhibition of mHS plays an important physiological role in suppressing hepatic ketogenesis during the starved to fed transition and in response to high carbohydrate feeding. Second, succinyl-CoA reacts with the ketolytic enzyme, SCOT, in converting AcAc to AcAc—CoA. Thus, high succinyl-CoA levels favor diversion of AcAc towards oxidation and away from the βHBD reaction. Finally, because succinyl-CoA also functions as a TCA cycle intermediate, its depletion can impede oxidative flux and force accumulation of acetyl-CoA. High ketone levels have been shown to lower succinyl-CoA levels by inhibiting it production via the αketoglutarate dehydrogenase complex (αKGD). Thus, elevations in intramuscular succinyl-CoA levels can oppose βHB accumulation and promote insulin sensitivity. Illustrative therapeutic strategies for reducing ketones in skeletal muscle and/or treating insulin resistance (including diabetes) include supplying exogenous succinate esters to skeletal muscle, which can be used by succinate thiokinase to generate succinyl-CoA, and/or provision of succinate precursors such as glutamate to skeletal muscle.

Alternatively, the invention provides methods of reducing ketone levels in skeletal muscle and/or treating insulin resistance (including diabetes) by delivering an isolated nucleic acid encoding succinate thiokinase to skeletal muscle such that the activity of succinate thiokinase in skeletal muscle is enhanced. The nucleic acid and amino acid sequences of succinate thiokinase are known in the art (see, e.g., Accession Number NM_{—}003849 [Homo sapiens; GDP-forming, alpha subunit]; Accession No. NM_{—}003848 [Homo sapiens, GDP-forming, beta subunit]; Accession No. AF104921 [Homo sapiens; alpha subunit]; Accession No. NM_{—}003850 [Homo sapiens; ADP-forming, beta subunit]).

The present invention further provides methods of lowering or normalizing skeletal muscle ketone levels by reducing the activity of ketogenic enzymes in skeletal muscle and/or liver. Ketogenic enzyme activity can be reduced by any method known in the art, which can be achieved at the nucleic acid and/or protein level.

In representative embodiments of the invention, the invention provides a method of reducing ketone levels in a skeletal muscle cell by contacting the skeletal muscle cell with an inhibitory oligonucleotide or a delivery vector that encodes an inhibitory oligonucleotide in an amount effective to reduce ketone levels in skeletal muscle. Inhibitory oligonucleotides can be RNA, DNA, or chimerics thereof and can further include non-naturally occurring nucleotides, sugars or linkages. Exemplary “inhibitory oligonucleotides” include antisense and RNA interference (RNAi) molecules, as well as ribozymes, external guide sequence oligonucleotides, and other short catalytic oligonucleotides that hybridize to the target sequence and reduce production of enzyme. In other approaches, enzyme activity is reduced using antibodies directed against the enzyme that inhibit the activity thereof, increase the turnover of the enzyme, or both.

Ribozymes are RNA-protein complexes that cleave nucleic acids in a site-specific fashion. Ribozymes have specific catalytic domains that possess endonuclease activity (Kim et al., (1987) Proc. Natl. Acad. Sci. USA 84:8788; Gerlach et al., (1987) Nature 328:802; Forster and Symons, (1987) Cell 49:211). For example, a large number of ribozymes accelerate phosphoester transfer reactions with a high degree of specificity, often cleaving only one of several phosphoesters in an oligonucleotide substrate (Michel and Westhof, (1990) J. Mol. Biol. 216:585; Reinhold-Hurek and Shub, (1992) Nature 357:173). This specificity has been attributed to the requirement that the substrate bind via specific base-pairing interactions to the internal guide sequence (“IGS”) of the ribozyme prior to chemical reaction.

By “specifically hybridize” (or grammatical variations) it is meant that there is a sufficient degree of complementarity or precise pairing between the inhibitory oligonucleotide and the target nucleic acid such that stable and specific binding occurs between the oligonucleotide and the target. It is understood in the art that the sequence of the inhibitory oligonucleotide need not be 100% complementary to that of its target nucleic acid to be specifically hybridizable. An inhibitory oligonucleotide is specifically hybridizable when binding of the oligonucleotide to the target nucleic acid interferes with the normal function of the target nucleic acid (e.g., replication, transcription and/or translation), and there is a sufficient degree of complementarity to avoid non-specific binding of the inhibitory oligonucleotide to non-target nucleic acids under conditions in which specific binding is desired, e.g., under physiological conditions in the case of in vivo assays or therapeutic treatment and in the case of in vitro assays, under conditions in which the assays are performed. As is known in the art, a higher degree of sequence similarity is generally required for short oligonucleotides, whereas a greater degree of mismatched bases will be tolerated by longer oligonucleotides.

As discussed in more detail below, the inhibitory oligonucleotide can be synthesized in vitro, for example, by chemical synthesis or transcription from an expression vector. The inhibitory oligonucleotide can be introduced into cells using transfection, electroporation or other techniques known in the art. Alternatively, the inhibitory oligonucleotide can be introduced using a lipid based delivery vector (discussed in more detail below).

In another approach, the inhibitory oligonucleotide can be generated in vivo in a cell after delivery and expression from a delivery vector encoding the inhibitory oligonucleotide.

Ribozyme catalysis has primarily been observed as part of sequence-specific cleavage/ligation reactions involving nucleic acids (Joyce, (1989) Nature 338:217). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of nucleic acid expression may be particularly suited to therapeutic applications. (Scanlon et al., (1991) Proc. Natl. Acad. Sci. USA 88:10591; Sarver et al., (1990) Science 247:1222; Sioud et al., (1992) J. Mol. Biol. 223:831).

In a representative embodiment, the invention provides a method of treating insulin resistance or diabetes (in particular, type 2 diabetes) comprising administering a pharmaceutical formulation comprising an inhibitory oligonucleotide or a delivery vector comprising a nucleic acid encoding an inhibitory oligonucleotide that specifically hybridizes to a target sequence encoding a ketogenic enzyme operably linked to a control element that directs expression of the nucleic acid in skeletal muscle in a therapeutically effective amount to reduce skeletal muscle ketone levels.

Illustrative ketogenic enzymes include but are not limited to β-hydroxybutyrate dehydrogenase, mitochondrial HMG-CoA synthase, acetoacetyl-CoA thiolase, and HMG-CoA lyase.

β-hydroxybutyrate dehydrogenase (EC 1.1.1.30) is encoded by the βDH gene and is a lipid-requiring mitochondrial membrane enzyme. This protein has a specific requirement for phosphatidylcholine for optimal enzymatic function and is a member of the short-chain alcohol dehydrogenase superfamily. Nucleic acid and amino acid sequences of β-hydroxybutyrate dehydrogenase are known in the art (see, e.g., Accession No. NM_{—}004051; Marks, et al. (1992) J. Biol. Chem. 267(22):15459-15463).

Mitochondrial HMG CoA synthase (EC 2.3.3.10) is the first enzyme in the ketogenic pathway, whereas the cytoplasmic isozyme mediates an early step in cholesterol synthesis. Mitochondrial HMG CoA synthase catalyzes the condensation of acetyl-CoA with acetoacetyl-CoA to form HMG CoA and CoA. The nucleic acid and amino acid sequences of mitochondrial HMG CoA synthase are known in the art (see, e.g., Accession No. NM_{—}005518, Boukaftane, et al. (1994) Genomics 23(3):552-559; and Accession No. L25798, Rokosz et al. 1994) Arch. Biochem. Biophys. 312:1-13).

Acetoacetyl-CoA thiolase (ACAT1; also known as acetyl-Coenzyme A acetyltransferase, β-ketothiolase, and 3-ketoacyl-CoA thiolase) is a mitochondrially localized enzyme that catalyzes the reversible formation of acetoacetyl-CoA from two molecules of acetyl-CoA. The ACAT1 gene spans approximately 27 kb and contains 12 exons interrupted by 11 introns. Defects in this gene are associated with the alpha-methylacetoaceticaciduria disorder, an inborn error of isoleucine catabolism characterized by urinary excretion of 2-methyl-3-hydroxybutyric acid, 2-methylacetoacetic acid, tiglylglycine, and butanone. The nucleic acid and amino acid sequences of ACAT1 are known (see, ACAT1, Fukao, et al. (1990) J. Clin. Invest. 86(6):2086-2092).

HMG-CoA lyase (hydroxymethylglutaryl-CoA lyase, EC 4.1.3.4) catalyzes the conversion of (S)-3-hydroxy-3-methylglutaryl-CoA to acetyl-CoA +acetoacetate. The enzyme is dually localized in the mitochondria and peroxisome, and contains a 27-residue N-terminal mitochondrial targeting sequence which in cleaved on mitochondrial entry, as well as a C-terminal Cys-Lys-Leu peroxisomal targeting motif. The mitochondrial enzyme (approximately 31 kDa) catalyzes the last step of ketogenesis; the function of the peroxisomal localized enzyme (approximately 33.5 kDa) is unknown. For illustrative nucleic acid and amino acid sequences for HMG-CoA lyase, see Accession No. L07033 (Mitchell, et al. (1993) J. Biol. Chem. 268(6):4376-4381).

In other embodiments, lipid oxidation is increased by suppression of acetyl CoA carboxylase (ACC), for example, in liver, skeletal muscle and/or adipose tissue. This can be achieved by direct suppression of ACC itself (e.g., with an inhibitory oligonucleotide as discussed above) or via an increase in 5′ AMP kinase activity, which causes phosphorylation and inactivation of ACC.

Acetyl CoA carboxylase (ACC; EC 6.4.1.2) is a complex multifunctional enzyme system. ACC1, also known as ACC-α, is a cytosolic enzyme, enriched in liver, adipose tissue and lactating mammary tissues. ACC1 is a biotin-containing enzyme which catalyzes the carboxylation of acetyl-CoA to malonyl-CoA, the rate-limiting step in fatty acid synthesis. ACC1 carries three functions: biotin carboxyl carrier protein, biotin carboxylase, and carboxyltransferase (catalytic activity). Variants of ACC1 have been described: one with eight additional amino acids commencing at Pro-1196, and the other which is 59 amino acids shorter than the predominant fat and liver isoform existing in mammals. The two ACC1 isoforms are differentially regulated in a tissue specific manner and under different physiological conditions. The activity of ACC1 is finely regulated by hormone-dependent phosphorylation and dephosphorylation. ACC-2, also known as ACC-β, is predominantly present in heart and skeletal muscle and to a lesser extent in liver. In contrast with ACC-1, which is cytosolic and catalyzes only fatty acid synthesis, ACC-2 co-localizes with carnitine palmitoyl transferase 1 (CPT-1) in the contact sites of the mitochondrial membranes. CPT-1 is potently inhibited by the lipogenic precursor malonyl CoA. Suppression of ACC activity lowers malonyl CoA levels, thereby increasing the catalytic activity of CPT-1, and in turn, the rate of fatty acid oxidation. ACC-2 contains a unique 114 amino acid long N-terminal peptide, accounting in part, for its regulatory role in fatty acid oxidation. Sequences of various ACC enzymes are known in the art, e.g., Accession No. AJ575592 (ACC2; Ha et al. (1996) Proc. Natl. Acad. Sc. U.S.A. 93:11466-11470), Accession No. AY315627 (ACC1, alternatively spliced; Mao et al. (2003) Proc. Natl. Acad. Sci. U.S.A. 100:7515-7520), Accession No. AY315626 (truncated ACC1 isoform; Mao et al. (2003) Proc. Natl. Acad. Sci. U.S.A. 100:7515-7520).

Those skilled in the art will appreciate that the isolated nucleic acids (e.g., encoding an inhibitory oligonucleotide, ketolytic or lipid oxidizing enzyme) of the invention are typically associated with appropriate expression control sequences, e.g., transcription/translation control signals and polyadenylation signals.

A variety of promoter/enhancer elements can be used depending on the level and tissue-specific expression desired. The promoter can be constitutive or inducible (e.g., the metalothionein promoter or a hormone inducible promoter), depending on the pattern of expression desired. The promoter can be native or foreign and can be a natural or a partially or completely synthetic sequence. By foreign, it is intended that the transcriptional initiation region is not found in the wild-type host into which the transcriptional initiation region is introduced. The promoter is chosen so that it will function in the target cell(s) of interest.

In particular embodiments, the nucleic acid is operably linked with a control element (e.g., a promoter) that directs the expression of the nucleic acid in liver (e.g., liver parenchyma) and/or in skeletal muscle. Further, the control element can express the nucleic acid specifically or preferentially in liver or skeletal muscle.

To illustrate, control elements that preferentially or specifically direct expression in liver include but are not limited to: a liver cell-specific human alphal-antitrypsin (hAAT) promoter, liver-specific transthyretin promoter (HD-IFN) (Aurisicchio et al. (2000), J. Virol. 74(10):4816-23), phosphoenol pyruvate carboxykinase (PEPCK) promoter (Haas, et al. (1999) Am. J. Pathol. 155(1):183-92), ornithine transcarbamylase (OTC) promoter (Murakami, et al. (1989) Dev. Genet 10(5):393401), albumin gene promoter/enhancer (alb e/p) (Miyatake, et al. (1999) Gene Ther. 6(4):564-72) and chimeric constructs combining promoter and enhancer regions of the albumin, alpha1-antitrypsin, hepatitis B virus core protein, and hemopexin genes (Kramer, et al. (2003) Mol. Ther. 7(3):375-85).

Control elements that preferentially or specifically direct expression in skeletal muscle include but are not limited to: the 5′ enhancer of the MCK gene (Jaynes, et al. (1988) Mol. Cell. Biol. 8:62-70), MLC1f promoter with the MLC⅓ 3′ enhancer (Donoghue, et al. (1991) J. Cell. Biol. 115:423-34), and the alpha-skeletal actin promoter (Brennan and Hardeman (1993) J. Biol. Chem. 268(1):719-25).

Moreover, specific initiation signals are generally used for efficient translation of inserted protein coding sequences. These translational control sequences, which can include the ATG initiation codon and adjacent sequences, can be of a variety of origins, both natural and synthetic.

In embodiments of the invention, the isolated nucleic acid comprises two or more heterologous nucleic acid sequences, the transcriptional units can be operatively associated with separate promoters or with a single upstream promoter and one or more downstream internal ribosome entry site (IRES) sequences (e.g., the picornavirus EMC IRES sequence).

The isolated nucleic acids can be incorporated into a vector, e.g., for the purposes of cloning or other laboratory manipulations, recombinant protein or oligonucleotide production, or delivery to a cell. Exemplary vectors include bacterial artificial chromosomes, cosmids, yeast artificial chromosomes, phage, plasmids, lipid vectors and viral vectors. Viral and nonviral delivery vectors are described in more detail below.

The present invention further provides cells comprising the nucleic acids, e.g., for use in producing inhibitory oligonucleotides in vitro or for the screening methods of the invention (described below).

III. Antisense Oligonucleotides

The sequences of ketogenic enzymes and ACC enzymes from a variety of sources are known (e.g., see above) and an antisense oligonucleotide or nucleic acid encoding an antisense oligonucleotide can be generated to any portion thereof in accordance with known techniques.

The term “antisense oligonucleotide,” as used herein, refers to a nucleic acid that is complementary to a specified DNA or RNA sequence. Antisense oligonucleotides and nucleic acids that encode the same can be made in accordance with conventional techniques. See, e.g., U.S. Pat. No. 5,023,243 to Tullis; U.S. Pat. No. 5,149,797 to Pederson et al.

Those skilled in the art will appreciate that it is not necessary that the antisense oligonucleotide be fully complementary to the target sequence as long as the degree of sequence similarity is sufficient for the antisense nucleotide sequence to specifically hybridize to its target (as defined above) and reduce production of the enzyme (e.g., by at least about 30%, 40%, 50%, 60%, 70%, 80%, 90%, 95%. or more).

For example, hybridization of such oligonucleotides to target sequences can be carried out under conditions of reduced stringency, medium stringency or even stringent conditions (e.g., conditions represented by a wash stringency of 35-40% Formamide with 5× Denhardt's solution, 0.5% SDS and 1×SSPE at 37° C.; conditions represented by a wash stringency of 40-45% Formamide with 5× Denhardt's solution, 0.5% SDS, and 1×SSPE at 42° C.; and/or conditions represented by a wash stringency of 50% Formamide with 5× Denhardt's solution, 0.5% SDS and 1×SSPE at 42° C., respectively). See, e.g., Sambrook et al., Molecular Cloning, A Laboratory Manual (2d Ed. 1989) (Cold Spring Harbor Laboratory).

Alternatively stated, in particular embodiments, antisense oligonucleotides of the invention have at least about 60%, 70%, 80%, 90%, 95%, 97%, 98% or higher sequence similarity with the complement of the target sequence and reduces enzyme production (as defined above). In some embodiments, the antisense sequence contains 1, 2, 3, 4, 5, 6, 7, 8, 9 or 10 mismatches as compared with the target sequence.

As is known in the art, a number of different programs can be used to identify whether a nucleic acid or polypeptide has sequence similarity to a known sequence. Sequence similarity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol. Biol. 48,443 (1970), by the search for similarity method of Pearson & Lipman, Proc. Natl. Acad. Sci. USA 85,2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12, 387-395 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35, 351-360 (1987); the method is similar to that described by Higgins & Sharp, CABIOS 5, 151-153 (1989).

Another example of a useful algorithm is the BLAST algorithm, described in Altschul et al., J. Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460480 (1996); http:H/blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., (1997) Nucleic Acids Res. 25, 3389-3402.

The length of the antisense oligonucleotide is not critical as long as it specifically hybridizes to the intended target and reduces enzyme production (as defined above) and can be determined in accordance with routine procedures. In general, the antisense oligonucleotide is from about eight, ten or twelve nucleotides in length and/or less than about 20, 30, 40, 50, 60, 70, 80, 100 or 150 nucleotides in length.

An antisense oligonucleotide can be constructed using chemical synthesis and enzymatic ligation reactions by procedures known in the art. For example, an antisense oligonucleotide can be chemically synthesized using naturally occurring nucleotides or various modified nucleotides designed to increase the biological stability of the molecules and/or to increase the physical stability of the duplex formed between the antisense and sense nucleotide sequences, e.g., phosphorothioate derivatives and acridine substituted nucleotides can be used.

Examples of modified nucleotides which can be used to generate the antisense oligonucleotide include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomet-hyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopenten-yladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotides of the invention further include nucleotide sequences wherein at least one, or all, or the internucleotide bridging phosphate residues are modified phosphates, such as methyl phosphonates, methyl phosphonothioates, phosphoromorpholidates, phosphoropiperazidates and phosphoramidates. For example, every other one of the internucleotide bridging phosphate residues can be modified as described.

In another non-limiting example, one or all of the nucleotides in the oligonucleotide contain a 2′ loweralkyl moiety (e.g., C₁-C₄, linear or branched, saturated or unsaturated alkyl, such as methyl, ethyl, ethenyl, propyl, 1-propenyl, 2-propenyl, and isopropyl). For example, every other one of the nucleotides can be modified as described. See also, Furdon et al., (1989) Nucleic Acids Res. 17, 9193-9204; Agrawal et al., (1990) Proc. Natl. Acad. Sci. USA 87, 1401-1405; Baker et al., (1990) Nucleic Acids Res. 18, 3537-3543; Sproat et al., (1989) Nucleic Acids Res. 17, 3373-3386; Walder and Walder, (1988) Proc. Natl. Acad. Sci. USA 85, 5011-5015; incorporated by reference herein in their entireties for their teaching of methods of making antisense molecules, including those containing modified nucleotide bases).

The antisense oligonucleotide can be chemically modified (e.g., at the 3′ or 5′ end) to be covalently conjugated to another molecule. To illustrate, the antisense oligonucleotide can be conjugated to a molecule that facilitates delivery to a cell of interest (e.g., liver or skeletal muscle cell), provides a detectable marker, increases the bioavailability of the oligonucleotide, increases the stability of the oligonucleotide, improves the formulation or pharmacokinetic characteristics, and the like. Examples of conjugated molecules include but are not limited to cholesterol, lipids, polyamines, polyamides, polyesters, intercalators, reporter molecules, biotin, dyes, polyethylene glycol, human serum albumin, an enzyme, an antibody or antibody fragment, or a ligand for a cellular receptor.

Other modifications to nucleic acids to improve the stability, nuclease-resistance, bioavailability, formulation characteristics and/or pharmacokinetic properties are known in the art.

Chemically synthesized oligonucleotides can be administered directly to a cell or subject. Alternatively, the antisense oligonucleotide can be produced using an expression vector into which a nucleic acid has been cloned in an antisense orientation. The antisense oligonucleotide can be expressed from the vector in vitro or following administration in vivo.

IV. RNA Interference.

RNA interference (RNAi) provides another approach for modulating enzyme activity. RNAi is a mechanism of post-transcriptional gene silencing in which double-stranded RNA (dsRNA) corresponding to a target sequence of interest is introduced into a cell or an organism, resulting in degradation of the corresponding mRNA. The mechanism by which RNAi achieves gene silencing has been reviewed in Sharp et al, (2001) Genes Dev 15: 485-490; and Hammond et al., (2001) Nature Rev Gen 2:110-119). The RNAi effect persists for multiple cell divisions before gene expression is regained. RNAi is therefore a powerful method for making targeted knockouts or “knockdowns” at the RNA level. RNAi has proven successful in human cells, including human embryonic kidney and HeLa cells (see, e.g., Elbashir et al., Nature (2001) 411:494-8).

Initial attempts to use RNAi in mammalian cells resulted in antiviral defense mechanisms involving PKR in response to the dsRNA molecules (see, e.g., Gil et al. (2000) Apoptosis 5:107). It has since been demonstrated that short synthetic dsRNA of about 21 nucleotides, known as “short interfering RNAs” (siRNA) can mediate silencing in mammalian cells without triggering the antiviral response (see, e.g., Elbashir et al., Nature (2001) 411:494-8; Caplen et al., (2001) Proc. Nat. Acad. Sci. 98:9742).

In one embodiment, RNAi molecules (including siRNA molecules) can be expressed from nucleic acid expression vectors in vitro or in vivo as short hairpin RNAs (shRNA; see Paddison et al., (2002), PNAS USA 99:1443-1448), which are believed to be processed in the cell by the action of the RNase III like enzyme Dicer into 20-25 mer siRNA molecules. The shRNAs generally have a stem-loop structure in which two inverted repeat sequences are separated by a short spacer sequence that loops out. There have been reports of shRNAs with loops ranging from 3 to 23 nucleotides in length. The loop sequence is generally not critical. Exemplary loop sequences include the following motifs: AUG, CCC, UUCG, CCACC, CTCGAG, MGCUU, CCACACC and UUCMGAGA.

The RNAi can further comprise a circular molecule comprising sense and antisense regions with two loop regions on either side to form a “dumbbell” shaped structure upon dsRNA formation between the sense and antisense regions. This molecule can be processed in vitro or in vivo to release the dsRNA portion, e.g., a siRNA.

International patent publication WO 01/77350 describes a vector for bi-directional transcription to generate both sense and antisense transcripts of a heterologous sequence in a eukaryotic cell. This technique can be used to produce RNAi for use according to the invention.

Shinagawa et al. (2003) Genes & Dev. 17:1340 reported a method of expressing long dsRNAs from a CMV promoter (a pol II promoter), which method is also applicable to tissue specific pol II promoters. Likewise, the approach of Xia et al., (2002) Nature biotech. 20:1006, avoids poly(A) tailing and can be used in connection with tissue-specific promoters.

Methods of generating RNAi include chemical synthesis, in vitro transcription, digestion of long dsRNA by Dicer (in vitro or in vivo), expression in vivo from a delivery vector, and expression in vivo from a PCR-derived RNAi expression cassette (see, e.g., TechNotes 10(3) “Five Ways to Produce siRNAs,” from Ambion, Inc., Austin Tex.; available at www.ambion.com).

Guidelines for designing siRNA molecules are available (see e.g., literature from Ambion, Inc., Austin Tex.; available at www.ambion.com). In particular embodiments, the siRNA sequence has about 30-50% G/C content. Further, long stretches of greater than four T or A residues are generally avoided if RNA polymerase III is used to transcribe the RNA. Online siRNA target finders are available, e.g., from Ambion, Inc. (www.ambion.com), through the Whitehead Institute of Biomedical Research (www.jura.wi.mit.edu) or from Dharmacon Research, Inc. (www.dharmacon.com/).

According to the present invention, the dsRNA portion of the RNAi molecule is generally at least about 6, 8, 10 or 12 basepairs in length and/or less than about 16, 18, 19, 21, 23, 25, 27, 28, 29, 30, 31, 32, 33, 34 or 35 basepairs in length. In illustrative embodiments, the dsRNA is from about 19 to about 23, 25 or 29 basepairs in length. In other representative embodiments, the RNAi includes a short overhang (e.g., 1, 2, 3, 4, 5 or 6 bases) at each end. In particular embodiments, the RNAi comprises a 3′ dinucleotide (e.g., UU) overhang. The overhang(s) can be complementary, but need not be, with the target sequence.

In other embodiments, a long dsRNA is used, which can be processed in vitro or in vivo (e.g., by Dicer) to form siRNA. According to this approach, the dsRNA can be at least about 35, 40, 50, 70, 85, 100 and/or less than about 200, 300, 400, 500, 1000, 2000 basepairs or more in length.

The antisense region of the RNAi molecule can be completely complementary to the target sequence, but need not be as long as it specifically hybridizes to the target sequence (as defined above) and reduces production of the target enzyme. In some embodiments, hybridization of such oligonucleotides to target sequences can be carried out under conditions of reduced stringency, medium stringency or even stringent conditions.

In other embodiments, the antisense region of the RNAi has at least about 60%, 70%, 80%, 90%, 95%, 97%, 98% or higher sequence similarity with the complement of the target sequence and reduces production of the target enzyme. In some embodiments, the antisense region contains 1, 2, 3, 4, 5, 6, 7, 8, 9 or 10 mismatches as compared with the target sequence. Mismatches are generally tolerated better at the ends of the dsRNA than in the center portion.

In particular embodiments, the RNAi is formed by intermolecular complexing between two separate sense and antisense molecules. The RNAi comprises a ds region formed by the intermolecular basepairing between the two separate strands. In other embodiments, the RNAi comprises a ds region formed by intramolecular basepairing within a single nucleic acid molecule comprising both sense and antisense regions, typically as an inverted repeat (e.g., a shRNA or other stem loop structure, or a circular RNAi molecule). The RNAi can further comprise a spacer region between the sense and antisense regions.

The RNAi molecule can contain modified sugars, nucleotides, backbone linkages and other modifications as described above for antisense oligonucleotides.

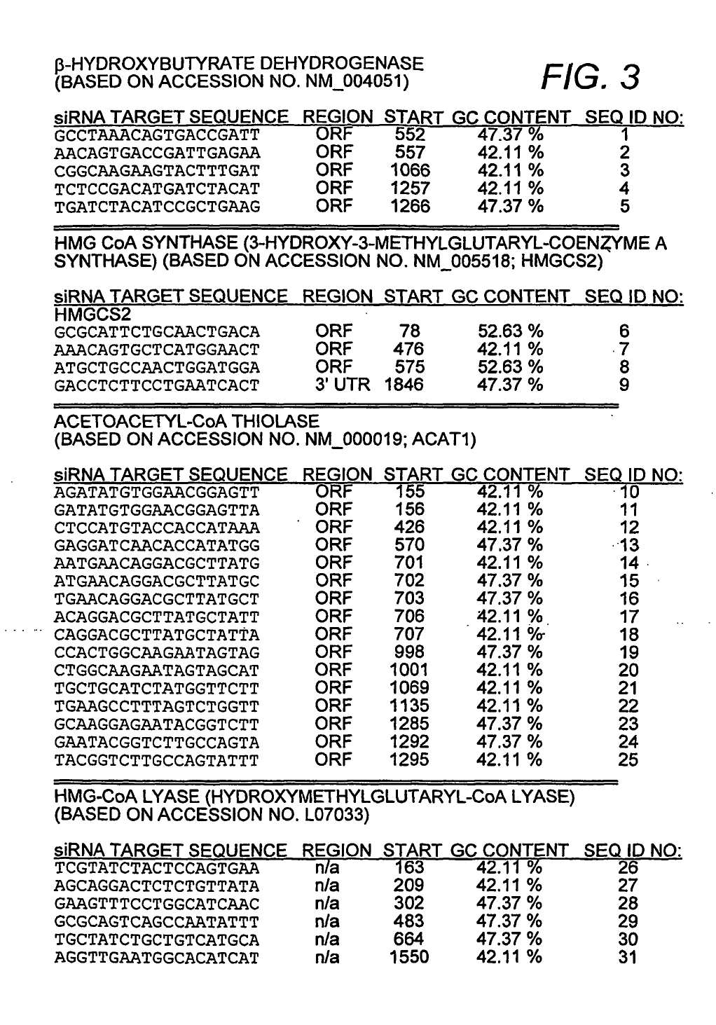

Exemplary target sequences against which an RNAi molecule can be directed for a variety of ketogenic enzymes are shown in FIG. 3.

Generally, RNAi molecules are highly selective. If desired, those skilled in the art can readily eliminate candidate RNAi that are likely to interfere with expression of nucleic acids other than the target by searching relevant databases to identify RNAi sequences that do not have substantial sequence homology with other known sequences, for example, using BLAST (available at www.ncbi.nim.nih.gov/BLAST).

Kits for the production of RNAi are commercially available, e.g., from New England Biolabs, Inc. and Ambion, Inc.

Silencing effects similar to those produced by RNAi have been reported in mammalian cells with transfection of a mRNA-cDNA hybrid construct (Lin et al., (2001) Biochem Biophys Res Commun 281:639-44), providing yet another strategy for silencing a coding sequence of interest.

V. Compounds for reducing Ketone Levels in Skeletal Muscle and Screening Methods.

The discovery that the concentration of ketones in skeletal muscle is correlated with skeletal muscle and whole animal insulin resistance provides new therapeutics and targets for drug discovery to treat insulin resistance or diabetes.

Thus, the invention provides a method of treating insulin resistance or diabetes comprising administering to a diabetic subject a compound that reduces skeletal muscle ketone levels in a therapeutically effective amount that reduces skeletal muscle ketone levels. To illustrate, the compound can enhance ketolytic activity in skeletal muscle, reduce ketogenic activity in skeletal muscle and/or enhance fatty acid oxidation in liver. The compound can interact directly with enzymes (or their coding sequences) within these metabolic pathways. Alternatively, the compound can interact with any other polypeptide, nucleic acid or other molecule if such interaction results in a reduction in skeletal muscle ketone levels.

In addition, the invention provides methods of identifying compounds for the treatment of insulin resistance or diabetes that modulate (i.e., enhance or reduce) the activity of enzymes involved in ketone synthesis or hydrolysis (e.g., by modulating the concentration or biological activity of the enzyme) at either the nucleic acid or protein level. Further, enzymes involved in lipid oxidation, which affect the availability of fatty acid precursors for ketogenesis, are targets to identify compounds for diabetes therapy or the treatment of insulin resistance. Accordingly, the invention also provides methods of identifying compounds for the treatment of diabetes and/or insulin resistance that modulate the activity (as these terms are defined above) of enzymes involved in fatty acid oxidation.

Any compound of interest can be administered or screened according to the present invention. Suitable compounds include small organic compounds (i.e., non-oligomers), oligomers or combinations thereof, and inorganic molecules. Suitable organic molecules can include but are not limited to polypeptides (including enzymes, antibodies and Fab′ fragments), carbohydrates, lipids, coenzymes, and nucleic acid molecules (including DNA, RNA and chimerics and analogs thereof) and nucleotides and nucleotide analogs. In particular embodiments, the compound is an antisense oligonucleotide, a RNAi or a ribozyme that inhibits production of the target enzyme.

Antisense oligonucleotides, RNAi and ribozymes are described in more detail above.

The compound can further be an antibody or antibody fragment. The antibody or antibody fragment can bind to the target enzyme (e.g., at the active site) and modulate the activity thereof. The term “antibody” or “antibodies” as used herein refers to all types of immunoglobulins, including IgG, IgM, IgA, IgD, and IgE. The antibody can be monoclonal or polyclonal and can be of any species of origin, including (for example) mouse, rat, rabbit, horse, or human, or can be a chimeric antibody. See, e.g., Walker et al., Molec. Immunol. 26, 403-11 (1989). The antibodies can be recombinant monoclonal antibodies produced according to the methods disclosed in U.S. Pat. No. 4,474,893 or U.S. Pat. No. 4,816,567. The antibodies can also be chemically constructed according to the method disclosed in U.S. Pat. No. 4,676,980.

Antibody fragments included within the scope of the present invention include, for example, Fab, F(ab′)2, and Fc fragments, and the corresponding fragments obtained from antibodies other than IgG. Such fragments can be produced by known techniques. For example, F(ab′)2 fragments can be produced by pepsin digestion of the antibody molecule, and Fab fragments can be generated by reducing the disulfide bridges of the F(ab′)2 fragments. Alternatively, Fab expression libraries can be constructed to allow rapid and easy identification of monoclonal Fab fragments with the desired specificity (Huse et al., (1989) Science 254,1275-1281).

Polyclonal antibodies used to carry out the present invention can be produced by immunizing a suitable animal (e.g., rabbit, goat, etc.) with an antigen to which a monoclonal antibody to the target binds, collecting immune serum from the animal, and separating the polyclonal antibodies from the immune serum, in accordance with known procedures.

Monoclonal antibodies used to carry out the present invention can be produced in a hybridoma cell line according to the technique of Kohler and Milstein, (1975) Nature 265, 495-97. For example, a solution containing the appropriate antigen can be injected into a mouse and, after a sufficient time, the mouse sacrificed and spleen cells obtained. The spleen cells are then immortalized by fusing them with myeloma cells or with lymphoma cells, typically in the presence of polyethylene glycol, to produce hybridoma cells. The hybridoma cells are then grown in a suitable medium and the supernatant screened for monoclonal antibodies having the desired specificity. Monoclonal Fab fragments can be produced in bacteria such as E. coli by recombinant techniques known to those skilled in the art. See, e.g., W. Huse, (1989) Science 246, 1275-81.

Antibodies specific to the target polypeptide can also be obtained by phage display techniques known in the art.

Small organic compounds (or “non-oligomers”) include a wide variety of organic molecules, such as heterocyclics, aromatics, alicyclics, aliphatics and combinations thereof, comprising steroids, antibiotics, enzyme inhibitors, ligands, hormones, drugs, alkaloids, opioids, terpenes, porphyrins, toxins, catalysts, as well as combinations thereof.

Oligomers include oligopeptides, oligonucleotides, oligosaccharides, polylipids, polyesters, polyamides, polyurethanes, polyureas; polyethers, and poly (phosphorus derivatives), e.g. phosphates, phosphonates, phosphoramides, phosphonamides, phosphites, phosphinamides, etc., poly (sulfur derivatives) e.g., sulfones, sulfonates, sulfites, sulfonamides, sulfenamides, etc., where for the phosphorous and sulfur derivatives the indicated heteroatom are optionally bonded to C,H,N,O or S, and combinations thereof. Such oligomers may be obtained from combinatorial libraries in accordance with known techniques.

Further, the methods of the invention can be practiced to screen a compound library, e.g., a combinatorial chemical compound library (e.g., benzodiazepine libraries as described in U.S. Pat. No. 5,288,514; phosphonate ester libraries as described in U.S. Pat. No. 5,420,328, pyrrolidine libraries as described in U.S. Pat. Nos. 5,525,735 and 5,525,734, and diketopiperazine and diketomorpholine libraries as described in U.S. Pat. No. 5,817,751), a polypeptide library, a cDNA library, a library of antisense nucleic acids, and the like, or an arrayed collection of compounds such as polypeptide and nucleic acid arrays.

Screening assays can be carried out in a cell free system, in cultured cells or in animals (e.g., non-human mammals) including transgenic animals (e.g., non-human transgenic mammals), each as known in the art.

The invention also encompasses compounds identified by the screening methods described herein.

The compounds of the present invention can optionally be administered in conjunction with other therapeutic agents useful in the treatment of diabetes or obesity. For example, the compounds of the invention can be administered in conjunction with insulin therapy and/or hypoglycemic agents.

The additional therapeutic agents can be administered concurrently with the compounds of the invention. As used herein, the word “concurrently” means sufficiently close in time to produce a combined effect (that is, concurrently can be simultaneously, or it can be two or more events occurring within a short time period before or after each other).

In general, the screening methods of the invention are carried out to identify compounds that bind to and/or enhance the activity of ketolytic enzymes (e.g., skeletal muscle or hepatic ketolytic enzymes), bind to and/or enhance the activity of lipid oxidizing enzymes (e.g., lipid oxidizing enzymes in liver) and/or bind to and/or reduce the activity of ketogenic enzymes (e.g., skeletal muscle or hepatic ketolytic enzymes).

In one representative embodiment, the invention provides methods of screening test compounds to identify a test compound that binds to the target enzyme. Compounds that are identified as binding to the target enzyme can be subject to further screening (e.g., for modulation of enzyme activity and/or activity in reducing skeletal muscle ketone levels and/or insulin resistance) using the methods described herein or other suitable techniques.

Also provided are methods of screening compounds to identify those that modulate the activity of the target enzyme. Methods of assessing the activity of enzymes involved in ketone and lipid metabolism in animal tissues, cells, or cell-free preparations are standard in the art. Compounds that are identified as modulators of enzyme activity can optionally be further screened (e.g., for binding to the target enzyme and/or activity in reducing skeletal muscle ketone levels and/or insulin resistance) using the methods described herein or other suitable techniques. The compound can directly interact with the target enzyme and thereby modulate its activity. Alternatively, the compound can interact with any other polypeptide, nucleic acid or other molecule as long as the interaction results in a modulation of enzyme activity.

As another aspect, the invention provides a method of screening compounds for activity in reducing ketone levels in a cell that produces ketones (e.g., a skeletal muscle or liver cell). In one representative embodiment, the method comprises contacting a cell that produces ketones with a test compound; and detecting ketone levels produced by the cell (e.g., by detecting ketone levels in the cell), wherein a reduction in ketone levels identifies the compound as a candidate for the treatment of insulin resistance or diabetes. In particular embodiments, βHB concentrations are detected.

In other representative embodiments, the invention provides methods of identifying a compound that reduces the concentration of a ketogenic enzyme, the activity of the ketogenic enzyme and/or the level of mRNA encoding the ketogenic enzyme in a cell. According to this embodiment, a cell that produces a ketogenic enzyme is contacted with a compound and the concentration of the ketogenic enzyme, the activity of the ketogenic enzyme, and/or the mRNA levels encoding the ketogenic enzyme in the cell is detected, wherein a reduction in the level of any of these indicia of ketogenic capacity in the cell identifies the compound as a candidate for the treatment of insulin resistance or diabetes. The ketogenic enzyme can be endogenously produced in the cell. Alternatively or additionally, the cell can be modified to comprise an isolated nucleic acid encoding the enzyme. In particular embodiments, the cell is a skeletal muscle cell or a liver cell.

As yet another approach, the invention provides a method of identifying a compound that enhances the concentration of a ketolytic enzyme, the activity of the ketolytic enzyme and/or the level of mRNA encoding the ketolytic enzyme in a cell. According to this embodiment, a cell that produces a ketolytic enzyme is contacted with a compound and the concentration of the ketolytic enzyme, the activity of the ketolytic enzyme, and/or the mRNA levels encoding the ketolytic enzyme in the cell is detected, wherein an enhancement in the level of any of these indicia of ketolytic capacity in the cell identifies the compound as a candidate for the treatment of insulin resistance or diabetes. The ketolytic enzyme can be endogenously produced in the cell. Alternatively or additionally, the cell can be modified to comprise an isolated nucleic acid encoding the enzyme. In particular embodiments, the cell is a skeletal muscle cell or a liver cell.

Similar methods can be carried out to identify compounds that enhance the concentration of a lipid oxidizing enzyme, the activity of the lipid oxidizing enzyme and/or the level of mRNA encoding the lipid oxidizing enzyme in a cell. In particular embodiments, the cell is a liver cell. For example, in one embodiment, the invention provides a method of identifying a compound that enhances the concentration of an enzyme involved in fatty acid oxidation, the activity of the fatty acid oxidizing enzyme and/or the level of mRNA encoding the fatty acid oxidizing enzyme in a cell. According to this embodiment, a cell that produces an enzyme involved in fatty acid oxidation is contacted with a compound and the concentration of the enzyme, the activity of the enzyme, and/or the mRNA levels encoding the enzyme in the cell is detected, wherein an enhancement in the level of any of these indicia of fatty acid oxidation capacity in the cell identifies the compound as a candidate for the treatment of insulin resistance or diabetes. The fatty acid oxidizing enzyme can be endogenously produced in the cell. Alternatively or additionally, the cell can be modified to comprise an isolated nucleic acid encoding the enzyme.

The screening assay can be a cell-based or cell-free assay. Further, the enzyme can be free in solution, affixed to a solid support, expressed on a cell surface, or located within a cell.

With respect to cell-free binding assays, test compounds can be synthesized or otherwise affixed to a solid substrate, such as plastic pins, glass slides, plastic wells, and the like. For example, the test compounds can be immobilized utilizing conjugation of biotin and streptavidin by techniques well known in the art. The test compounds are contacted with enzyme and washed. Bound enzyme can be detected using standard techniques in the art (e.g., by radioactive or fluorescence labeling of the enzyme, by ELISA methods, and the like).

Alternatively, the target enzyme can be immobilized to a solid substrate and the test compounds contacted with the bound enzyme. Identifying those test compounds that bind to and/or modulate enzyme activity can be carried out with routine techniques. For example, the test compounds can be immobilized utilizing conjugation of biotin and streptavidin by techniques well known in the art. As another illustrative example, antibodies reactive with the enzyme can be bound to the wells of the plate, and the enzyme trapped in the wells by antibody conjugation. Preparations of test compounds can be incubated in the enzyme-presenting wells and the amount of complex trapped in the well can be quantified.

In another representative embodiment, a fusion protein can be provided which comprises a domain that facilitates binding of the protein to a matrix. For example, glutathione-S-transferase fusion proteins can be adsorbed onto glutathione sepharose beads (Sigma Chemical, St. Louis, Mo.) or glutathione derivatized microtitre plates, which are then combined with cell lysates (e.g., ³⁵S-labeled) and the test compound, and the mixture incubated under conditions conducive to complex formation (e.g., at physiological conditions for salt and pH). Following incubation, the beads are washed to remove any unbound label, and the matrix immobilized and radiolabel detected directly, or in the supernatant fraction after the complexes are dissociated. Alternatively, the complexes can be dissociated from the matrix, separated by SDS-PAGE, and the level of the enzyme found in the bead fraction quantified from the gel using standard electrophoretic techniques.

Another technique for compound screening provides for high throughput screening of compounds having suitable binding affinity to the polypeptide of interest, as described in published PCT application WO 84/03564. In this method, a large number of different test compounds are synthesized on a solid substrate, such as plastic pins or some other surface. The test compounds are reacted with the target enzyme and washed. Bound enzyme is then detected by methods known in the art. Purified enzyme can also be coated directly onto plates for use in the aforementioned drug screening techniques. Alternatively, non-neutralizing antibodies can be used to capture the peptide and immobilize it on a solid support.

With respect to cell-based assays, any suitable cell can be used including bacteria, yeast, insect cells (e.g., with a baculovirus expression system), avian cells, mammalian cells, or plant cells. With particular respect to mammalian cells, screening can advantageously be carried out with muscle and liver cells. In particular embodiments, the cell will be from a subject with insulin resistance and/or diabetes and/or an obese subject, including animal models of these disorders.

The screening assay can be used to detect compounds that bind to and/or modulate the activity of native enzyme (e.g., enzyme that is normally produced by the cell). Alternatively, the cell can be modified to express a recombinant enzyme. According to this embodiment, the cell can be transiently or stably transformed with a nucleic acid encoding the enzyme, but is preferably stably transformed, for example, by stable integration into the genome of the organism or by expression from a stably maintained episome (e.g., Epstein Barr Virus derived episomes).

In a cell-based assay, the compound to be screened can interact directly with the enzyme or coding sequence (e.g., bind to it) and modulate the activity thereof. Alternatively, the compound can interact with the substrate of the target enzyme and/or any other cellular component, interaction with which results in an indirect modulation of enzyme activity. Enzyme activity can be modulated by effecting a change in the biological activity of the enzyme and/or stability of the polypeptide. Alternatively, the compound can be one that modulates enzyme activity at the nucleic acid level. To illustrate, the compound can modulate transcription of the gene encoding the enzyme (or transgene), modulate the accumulation of mRNA (e.g., by affecting the rate of transcription and/or turnover of the mRNA), and/or modulate the rate and/or amount of translation of the mRNA transcript.

As a further type of cell-based binding assay, the target enzyme can be used as a “bait protein” in a two-hybrid or three-hybrid assay (see, e.g., U.S. Pat. No. 5,283,317; Zervos et al., (1993) Cell 72:223-232; Madura et al., (1993) J. Biol. Chem. 268:12046-12054; Bartel et al., (1993) Biotechniques 14:920-924; Iwabuchi et al., (1993) Oncogene 8:1693-1696; and PCT publication WO94/10300), to identify other polypeptides that bind to or interact with the target enzyme.

The two-hybrid system is based on the modular nature of most transcription factors, which consist of separable DNA-binding and activation domains. Briefly, the assay utilizes two different DNA constructs. In one construct, the nucleic acid that encodes the target enzyme is fused to a nucleic acid encoding the DNA binding domain of a known transcription factor (e.g., GAL-4). In the other construct, a DNA sequence, optionally from a library of DNA sequences, that encodes an unidentified protein (“prey” or “sample”) is fused to a nucleic acid that codes for the activation domain of the known transcription factor. If the “bait” and the “prey” proteins are able to interact in vivo, forming a complex, the DNA-binding and activation domains of the transcription factor are brought into close proximity. This proximity allows transcription of a reporter sequence (e.g., LacZ), which is operably linked to a transcriptional regulatory'site responsive to the transcription factor. Expression of the reporter can be detected and cells containing the functional transcription factor can be isolated and used to obtain the nucleic acid encoding the polypeptide that exhibited binding to the target enzyme.

Screening assays can also be carried out in vivo in animals (e.g., non-human mammals). The enzyme activity can be based on endogenous enzyme levels and/or levels expressed from an isolated nucleic acid encoding the enzyme introduced into the animal. Thus, as still a further aspect, the invention provides a transgenic animal comprising an isolated nucleic acid encoding a target enzyme, which can be produced according to methods well-known in the art. The transgenic animal (e.g., a transgenic non-human mammal) can be any species, including avians and non-human mammals. According to this aspect of the invention, suitable non-human mammals include mice, rats, rabbits, guinea pigs, goats, sheep, pigs and cattle. Mammalian models for insulin resistance, obesity and/or diabetes can also be used (e.g., STZ diabetic mice, ob/ob mice). Suitable avians include chickens, ducks, geese, quail, turkeys and pheasants.

The nucleic acid encoding the target enzyme is stably incorporated into cells within the transgenic animal (typically, by stable integration into the genome or by stably maintained episomal constructs). It is not necessary that every cell contain the transgene, and the animal can be a chimera of modified and unmodified cells, as long as a sufficient number of cells (e.g., liver and/or skeletal muscle cells) comprise and express the transgene so that the animal is a useful screening tool (e.g., so that administration of compounds that modulate enzyme activity give rise to a detectable modulation in enzyme activity and/or ketone concentrations).

In particular embodiments, it is desirable that the enzyme be operably associated with a promoter or other transcriptional regulatory element that is functional in skeletal muscle cells or liver cells or is even specific to these cells. For example, in particular embodiments, the animal comprises an isolated nucleic acid encoding a ketogenic or ketolytic enzyme that is, optionally, operably linked with a control element that directs expression, or even specifically directs expression, in skeletal muscle: Similarly, in other illustrative embodiments, the animal comprises an isolated nucleic acid encoding a lipid oxidizing enzyme that is, optionally, operably linked with a control element that directs expression, or even specifically directs expression, in the liver.

One exemplary method of identifying a candidate compound for the treatment of insulin resistance or diabetes comprises administering a compound to an animal, detecting skeletal muscle ketone levels in the animal, wherein a reduction in skeletal muscle ketone levels identifies the compound as a candidate for the treatment of insulin resistance or diabetes. In particular embodiments, βHB levels are detected.

As a further method of identifying a candidate compound for the treatment of insulin resistance or diabetes, the invention provides a method comprising: administering a compound to an animal, detecting an indicia in skeletal muscle or liver selected from the group consisting of the concentration of a ketogenic enzyme, activity of a ketogenic enzyme and/or mRNA levels encoding a ketogenic enzyme, wherein a reduction in the level of the indicia of ketogenic activity in skeletal muscle or liver identifies the compound as a candidate for the treatment of insulin resistance or diabetes.

As yet another approach for identifying a candidate compound for the treatment of insulin resistance or diabetes, the invention provides a method comprising: administering a compound to an animal, detecting an indicia in skeletal muscle or liver selected from the group consisting of the concentration of a ketolytic enzyme, activity of a ketolytic enzyme and/or mRNA levels encoding a ketolytic enzyme, wherein an enhancement in the level of the indicia of ketolytic activity in skeletal muscle or liver identifies the compound as a candidate for the treatment of insulin resistance or diabetes.

Similar methods are provided for identifying a candidate compound for the treatment of insulin resistance or diabetes by detecting a compound that enhances the concentration of a lipid oxidizing enzyme, activity of a lipid oxidizing enzyme and/or mRNA levels encoding a lipid oxidizing enzyme in the liver, wherein an enhancement in the level of the indicia of lipid oxidation in the liver identifies the compound as a candidate for the treatment of insulin resistance or diabetes.

In still other embodiments, the invention provides a method of identifying a candidate compound for the treatment of insulin resistance or diabetes, comprising: administering a compound to a transgenic animal that exhibits insulin resistance, the transgenic animal comprising an isolated nucleic acid encoding a ketogenic enzyme, detecting the level of insulin resistance in the animal after administration of the compound, wherein a reduction in the level of insulin resistance identifies the compound as a candidate for the treatment of insulin resistance or diabetes. In exemplary methods, skeletal muscle insulin resistance is detected and a reduction in insulin resistance in skeletal muscle identifies the compound as a candidate for the treatment of diabetes.

Methods of making transgenic animals are known in the art. DNA constructs can be introduced into the germ line of an avian or mammal to make a transgenic animal. For example, one or several copies of the construct can be incorporated into the genome of an embryo by standard transgenic techniques.

In an exemplary embodiment, a transgenic animal is produced by introducing a transgene into the germ line of the animal. Transgenes can be introduced into embryonal target cells at various developmental stages. Different methods are used depending on the stage of development of the embryonal target cell. The specific line(s) of any animal used should, if possible, be selected for general good health, good embryo yields, good pronuclear visibility in the embryo, and good reproductive fitness.

Introduction of the transgene into the embryo can be accomplished by any of a variety of means known in the art such as microinjection, electroporation, lipofection or a viral vector. For example, the transgene can be introduced into a mammal by microinjection of the construct into the pronuclei of the fertilized mammalian egg(s) to cause one or more copies of the construct to be retained in the cells of the developing mammal(s). Following introduction of the transgene construct into the fertilized egg, the egg can be incubated in vitro for varying amounts of time, or reimplanted into the surrogate host, or both. One common method is to incubate the embryos in vitro for about 1-7 days, depending on the species, and then reimplant them into the surrogate host.

The progeny of the transgenically manipulated embryos can be tested for the presence of the construct by Southern blot analysis of a segment of tissue. An embryo having one or more copies of the exogenous cloned construct stably integrated into the genome can be used to establish a permanent transgenic animal line carrying the transgenically added construct.

Transgenically altered animals can be assayed after birth for the incorporation of the construct into the genome of the offspring. This can be done by hybridizing a probe corresponding to the DNA sequence coding for the polypeptide or a segment thereof onto chromosomal material from the progeny. Those progeny found to contain at least one copy of the construct in their genome are grown to maturity.

Methods of producing transgenic avians are also known in the art, see, e.g., U.S. Pat. No. 5,162,215.

VI. Delivery Vectors.

The methods of the present invention provide a means for delivering and expressing nucleic acids in both dividing and non-dividing cells in vitro or in vivo (e.g., in skeletal muscle or liver cells). In embodiments of the invention, the nucleic acid can be expressed transiently in the target cell or the nucleic acid can be stably incorporated into the target cell, for example, by integration into the genome of the cell or by persistent expression from stably maintained episomes (e.g., derived from Epstein Barr Virus).

As one aspect, the vectors, methods and pharmaceutical formulations of the present invention find use in a method of administering a nucleic acid encoding a ketolytic and/or lipid oxidizing enzyme to a subject in need thereof. The invention further finds use in methods of administering a nucleic acid comprising an inhibitory oligonucleotide or a nucleic acid encoding an inhibitory oligonucleotide to a subject in need thereof. Such subjects include subjects that are obese, are insulin resistant and/or are diabetic (e.g., type 2 diabetes). Pharmaceutical formulations and methods of delivering nucleic acids for therapeutic purposes are described in more detail below.

Nucleic acids encoding inhibitory oligonucleotides can be expressed transiently or stably in a cell culture system to produce the inhibitory oligonucleotides which are then administered to a cell or subject. Likewise, nucleic acids encoding ketogenic, ketolytic and/or lipid oxidizing enzymes can be expressed in culture for the purpose of screening assays (described herein). The cell can be a bacterial, protozoan, plant, yeast, fungus, or animal (e.g., insect, avian or mammalian) cell.

It will be apparent to those skilled in the art that any suitable vector can be used to deliver the isolated nucleic acids of this invention to the target cell(s) or subject of interest. The choice of delivery vector can be made based on a number of factors known in the art, including age and species of the target host, in vitro vs. in vivo delivery, level and persistence of expression desired, intended purpose (e.g., for therapy or drug screening), the target cell or organ, route of delivery, size of the isolated nucleic acid, safety concerns, and the like.

Suitable vectors include virus vectors (e.g., retrovirus, alphavirus; vaccinia virus; adenovirus, adeno-associated virus, or herpes simplex virus), lipid vectors, poly-lysine vectors, synthetic polyamino polymer vectors and the like.

Any viral vector that is known in the art can be used in the present invention. Examples of such viral vectors include, but are not limited to vectors derived from: Adenoviridae; Birnaviridae; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group Family ([PHgr]6 phage group; Cysioviridae; Group Carnation ringspot; Dianthovirus virus group; Group Broad bean wilt; Fabavirus virus group; Filoviridae; Flaviviridae; Furovirus group; Group Germinivirus; Group Giardiavirus; Hepadnaviridae; Herpesviridae; Hordeivirus virus group; Illarvirus virus group; Inoviridae; Iridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Pea enation mosaic virus group; Phycodnaviridae; Picomaviridae; Plasmaviridae; Prodoviridae; Polydnaviridae; Potexvirus group; Potyvirus; Poxviridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Torovirus; Totiviridae; Group Tymovirus; and Plant virus satellites.

Protocols for producing recombinant viral vectors and for using viral vectors for nucleic acid delivery can be found in Current Protocols in Molecular Biology, Ausubel, F. M. et al. (eds.) Greene Publishing Associates, (1989) and other standard laboratory manuals (e.g., Vectors for Gene Therapy. In: Current Protocols in Human Genetics. John Wiley and Sons, Inc.: 1997).

Particular examples of viral vectors are those previously employed for the delivery of nucleic acids including, for example, retrovirus, adenovirus, AAV, herpes virus, and poxvirus vectors.

In certain embodiments of the present invention, the delivery vector is an adenovirus vector. The term “adenovirus” as used herein is intended to encompass all adenoviruses, including the Mastadenovirus and Aviadenovirus genera. To date, at least forty-seven human serotypes of adenoviruses have been identified (see, e.g., FIELDS et al., VIROLOGY, volume 2, chapter 67 (3d ed., Lippincott-Raven Publishers). Preferably, the adenovirus is a serogroup C adenovirus, still more preferably the adenovirus is serotype 2 (Ad2) or serotype 5 (Ad5).

The various regions of the adenovirus genome have been mapped and are understood by those skilled in the art (see, e.g., FIELDS et al., VIROLOGY, volume 2, chapters 67 and 68 (3d ed., Lippincott-Raven Publishers). The genomic sequences of the various Ad serotypes, as well as the nucleotide sequence of the particular coding regions of the Ad genome, are known in the art and can be accessed, e.g., from GenBank and NCBI (see, e.g., GenBank Accession Nos. J0917, M73260, X73487, AF108105, L19443, NC 003266 and NCBI Accession Nos. NC 001405, NC 001460, NC 002067, NC 00454).

Those skilled in the art will appreciate that the inventive adenovirus vectors can be modified or “targeted” as described in Douglas et al., (1996) Nature Biotechnology 14:1574; U.S. Pat. No. 5,922,315 to Roy et al.; U.S. Pat. No. 5,770,442 to Wickham et al.; and/or U.S. Pat. No. 5,712,136 to Wickham et al.

An adenovirus vector genome or rAd vector genome will typically comprise the Ad terminal repeat sequences and packaging signal. An “adenovirus particle” or “recombinant adenovirus particle” comprises an adenovirus vector genome or recombinant adenovirus vector genome, respectively, packaged within an adenovirus capsid. Generally, the adenovirus vector genome is most stable at sizes of about 28 kb to 38 kb (approximately 75% to 105% of the native genome size). In the case of an adenovirus vector containing large deletions and a relatively small heterologous nucleic acid of interest, “stuffer DNA” can be used to maintain the total size of the vector within the desired range by methods known in the art.

Normally adenoviruses bind to a cell surface receptor (CAR) of susceptible cells via the knob domain of the fiber protein on the virus surface. The fiber knob receptor is a 45 kDa cell surface protein which has potential sites for both glycosylation and phosphorylation. (Bergelson et al., (1997), Science 275:1320-1323). A secondary method of entry for adenovirus is through integrins present on the cell surface. Arginine-Glycine-Aspartic Acid (RGD) sequences of the adenoviral penton base protein bind integrins on the cell surface.

The adenovirus genome can be manipulated such that it encodes and expresses a nucleic acid of interest but is inactivated in terms of its ability to replicate in a normal lytic viral life cycle. See, for example, Berkner et al. (1988) BioTechniques 6:616; Rosenfeld et al. (1991) Science 252:431-434; and Rosenfeld et al. (1992) Cell 68:143-155. Representative adenoviral vectors derived from the adenovirus strain Ad type 5 d1 324 or other strains of adenovirus (e.g., Ad2, Ad3, Ad7 etc.) are known to those skilled in the art.

Recombinant adenoviruses can be advantageous in certain circumstances in that they are not capable of infecting nondividing cells and can be used to infect a wide variety of cell types, including epithelial cells. Furthermore, the virus particle is relatively stable and amenable to purification and concentration, and can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., as occurs with retroviral DNA). Moreover, the carrying capacity of the adenoviral genome for foreign DNA is large relative to other delivery vectors (Haj-Ahmand and Graham (1986) J. Virol. 57:267).

In particular embodiments, the adenovirus genome contains a deletion therein, so that at least one of the adenovirus genomic regions does not encode a functional protein. For example, first-generation adenovirus vectors are typically deleted for the E1 genes and packaged using a cell that expresses the E1 proteins (e.g., 293 cells). The E3 region is also frequently deleted as well, as there is no need for complementation of this deletion. In addition, deletions in the E4, E2a, protein IX, and fiber protein regions have been described, e.g., by Armentano et al, (1997) J. Virology 71:2408, Gao et al., (1996) J. Virology 70:8934, Dedieu et al., (1997) J. Virology 71;4626, Wang et al., (1997) Gene Therapy 4:393, U.S. Pat. No. 5,882,877 to Gregory et al. (the disclosures of which are incorporated herein in their entirety). Preferably, the deletions are selected to avoid toxicity to the packaging cell. Wang et al., (1997) Gene Therapy 4:393, has described toxicity from constitutive co-expression of the E4 and E1 genes by a packaging cell line. Toxicity can be avoided by regulating expression of the E1 and/or E4 gene products by an inducible, rather than a constitutive, promoter. Combinations of deletions that avoid toxicity or other deleterious effects on the host cell can be routinely selected by those skilled in the art.

As further examples, in particular embodiments, the adenovirus is deleted in the polymerase (pol), preterminal protein (pTP), IVa2 and/or 100K regions (see, e.g., U.S. Pat. No. 6,328,958; PCT publication WO 00/12740; and PCT publication WO 02/098466; Ding et al., (2002) Mol. Ther. 5:436; Hodges et al., J. Virol. 75:5913; Ding et al., (2001) Hum Gene Ther 12:955; the disclosures of which are incorporated herein by reference in their entireties for the teachings of how to make and use deleted adenovirus vectors for gene delivery).

The term “deleted” adenovirus as used herein refers to the omission of at least one nucleotide from the indicated region of the adenovirus genome. Deletions can be greater than about 1, 2,3, 5, 10, 20, 50, 100, 200, or even 500 nucleotides. Deletions in the various regions of the adenovirus genome can be about at least 1%, 5%°, 10%, 25%, 50%, 75%, 90%, 95%, 99%, or more of the indicated region. Alternately, the entire region of the adenovirus genome is deleted. Preferably, the deletion will prevent or essentially prevent the expression of a functional protein from that region. In general, larger deletions are preferred as these have the additional advantage that they will increase the carrying capacity of the deleted adenovirus for a heterologous nucleotide sequence of interest. The various regions of the adenovirus genome have been mapped and are understood by those skilled in the art (see, e.g., FIELDS et al., VIROLOGY, volume 2, chapters 67 and 68 (3d ed., Lippincott-Raven Publishers).

Those skilled in the art will appreciate that typically, with the exception of the E3 genes, any deletions will need to be complemented in order to propagate (replicate and package) additional virus, e.g., by transcomplementation with a packaging cell.

The present invention can also be practiced with “gutted” adenovirus vectors (as that term is understood in the art, see e.g., Lieber et al., (1996) J. Virol. 70:8944-60) in which essentially all of the adenovirus genomic sequences are deleted.

Adeno-associated viruses (AAV) have also been employed as nucleic acid delivery vectors. For a review, see Muzyczka et al. Curr. Topics in Micro. and Immunol. (1992) 158:97-129). AAV are parvoviruses and have small icosahedral virions, 18-26 nanometers in diameter and contain a single stranded genomic DNA molecule 4-5 kilobases in size. The viruses contain either the sense or antisense strand of the DNA molecule and either strand is incorporated into the virion. Two open reading frames encode a series of Rep and Cap polypeptides. Rep polypeptides (Rep50, Rep52, Rep68 and Rep78) are involved in replication, rescue and integration of the AAV genome, although significant activity can be observed in the absence of all four Rep polypeptides. The Cap proteins (VP1, VP2, VP3) form the virion capsid. Flanking the rep and cap open reading frames at the 5′ and 3′ ends of the genome are 145 basepair inverted terminal repeats (ITRs), the first 125 basepairs of which are capable of forming Y- or T-shaped duplex structures. It has been shown that the ITRs represent the minimal cis sequences required for replication, rescue, packaging and integration of the AAV genome. Typically, in recombinant AAV vectors (rAAV), the entire rep and cap coding regions are excised and replaced with a heterologous nucleic acid of interest.

AAV are among the few viruses that can integrate their DNA into non-dividing cells, and exhibit a high frequency of stable integration into human chromosome 19 (see, for example, Flotte et al. (1992) Am. J. Respir. Cell. Mol. Biol. 7:349-356; Samulski et al., (1989) J Virol. 63:3822-3828; and McLaughlin et al., (1989) J. Virol. 62:1963-1973). A variety of nucleic acids have been introduced into different cell types using AAV vectors (see, for example, Hermonat et al., (1984) Proc. Natl. Acad. Sci. USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flotte et al., (1993) J. Biol. Chem. 268:3781-3790).

A rAAV vector genome will typically comprise the AAV terminal repeat sequences and packaging signal. An “AAV particle” or “rAAV particle” comprises an AAV vector genome or rAAV vector genome, respectively, packaged within an AAV capsid. The rAAV vector itself need not contain AAV genes encoding the capsid and Rep proteins. In particular embodiments of the invention, the rep and/or cap genes are deleted from the AAV genome. In a representative embodiment, the rAAV vector retains only the terminal AAV sequences (ITRs) necessary for integration, excision, replication.

Sources for the AAV capsid genes can include serotypes AAV-1, AAV-2, AAV-3 (including 3a and 3b), AAV-4, AAV-5, AAV-6, AAV-7, AAV-8, as well as. bovine AAV and avian AAV, and any other virus classified by the International Committee on Taxonomy of Viruses (ICTV) as an AAV (see, e.g., BERNARD N. FIELDS et al., VIROLOGY, volume 2, chapter 69 (4th ed., Lippincott-Raven Publishers).

Because of packaging limitations, the total size of the rAAV genome will preferably be less than about 5.2, 5, 4.8, 4.6 or 4.5 kb in size.

Any suitable method known in the art can be used to produce AAV vectors expressing the nucleic acids of this invention (see, e.g., U.S. Pat. No. 5,139,941; U.S. Pat. No. 5,858,775; U.S. Pat. No. 6,146,874 for illustrative methods). In one particular method, AAV stocks can be produced by co-transfection of a rep/cap vector encoding AAV packaging functions and the template encoding the AAV vDNA into human cells infected with the helper adenovirus (Samulski et al., (1989) J. Virology 63:3822).

In other particular embodiments, the adenovirus helper virus is a hybrid helper virus that encodes AAV Rep and/or capsid proteins. Hybrid helper Ad/AAV vectors expressing AAV rep and/or cap genes and methods of producing AAV stocks using these reagents are known in the art (see, e.g., U.S. Pat. No. 5,589,377; and U.S. Pat. No. 5,871,982, U.S. Pat. No. 6,251,677; and U.S. Pat. No. 6,387,368). Preferably, the hybrid Ad of the invention expresses the AAV capsid proteins (i.e., VP1, VP2, and VP3). Alternatively, or additionally, the hybrid adenovirus can express one or more of AAV Rep proteins (i.e., Rep40, Rep52, Rep68 and/or Rep78). The AAV sequences can be operatively associated with a tissue-specific or inducible promoter.

The AAV rep and/or cap genes can alternatively be provided by a packaging cell that stably expresses the genes (see, e.g., Gao et al., (1998) Human Gene Therapy 9:2353; Inoue et al., (1998) J. Virol. 72:7024; U.S. Pat. No. 5,837,484; WO 98/27207; U.S. Pat. No. 5,658,785; WO 96/17947).

Another vector for use in the present invention comprises Herpes Simplex Virus (HSV). Herpes simplex virions have an overall diameter of 150 to 200 nm and a genome consisting of one double-stranded DNA molecule that is 120 to 200 kilobases in length. Glycoprotein D (gD) is a structural component of the HSV envelope that mediates virus entry into host cells. The initial interaction of HSV with cell surface heparin sulfate proteoglycans is mediated by another glycoprotein, glycoprotein C (gC) and/or glycoprotein B (gB). This is followed by interaction with one or more of the viral glycoproteins with cellular receptors. It has been shown that glycoprotein D of HSV binds directly to Herpes virus entry mediator (HVEM) of host cells. HVEM is a member of the tumor necrosis factor receptor superfamily (Whitbeck et al., (1997), J. Virol.; 71:6083-6093). Finally, gD, gB and the complex of gH and gL act individually or in combination to trigger pH-independent fusion of the viral envelope with the host cell plasma membrane. The virus itself is transmitted by direct contact and replicates in the skin or mucosal membranes before infecting cells of the nervous system for which HSV has particular tropism. It exhibits both a lytic and a latent function. The lytic cycle results in viral replication and cell death. The latent function allows for the virus to be maintained in the host for an extremely long period of time.

HSV can be modified for the delivery of nucleic acids to cells by producing a vector that exhibits only the latent function for long-term gene maintenance. HSV vectors are useful for nucleic acid delivery because they allow for a large DNA insert of up to or greater than 20 kilobases; they can be produced with extremely high titers; and they have been shown to express nucleic acids for a long period of time in the central nervous system as long as the lytic cycle does not occur.

In other particular embodiments of the present invention, the delivery vector of interest is a retrovirus. Retroviruses normally bind to a virus-specific cell surface receptor, e.g., CD4 (for HIV); CAT (for MLV-E; ecotropic Murine leukemic virus E); RAM1/GLVR2 (for murine leukemic virus-A; MLV-A); GLVR1 (for Gibbon Ape leukemia virus (GALV) and Feline leukemia virus B (FeLV-B)). The development of specialized cell lines (termed “packaging cells”) which produce only replication-defective retroviruses has increased the utility of retroviruses for gene therapy, and defective retroviruses are characterized for use in gene transfer for gene therapy purposes (for a review, see Miller, (1990) Blood 76:271). A replication-defective retrovirus can be packaged into virions which can be used to infect a target cell through the use of a helper virus by standard techniques.

Yet another suitable vector is a poxvirus vector. These viruses are very complex, containing more than 100 proteins, although the detailed structure of the virus is presently unknown. Extracellular forms of the virus have two membranes while intracellular particles only have an inner membrane. The outer surface of the virus is made up of lipids and proteins that surround the biconcave core. Poxviruses are antigenically complex, inducing both specific and cross-reacting antibodies after infection. Poxvirus receptors are not presently known, but it is likely that there exists more than one given the tropism of poxvirus for a wide range of cells. Poxvirus gene expression is well studied due to the interest in using vaccinia virus as a vector for expression of nucleic acids.

In addition to viral transfer methods, such as those illustrated above, non-viral methods can also be employed. Many non-viral methods of nucleic acid transfer rely on normal mechanisms used by mammalian cells for the uptake and intracellular transport of macromolecules. In particular embodiments, non-viral nucleic acid delivery systems rely on endocytic pathways for the uptake of the nucleic acid molecule by the targeted cell. Exemplary nucleic acid delivery systems of this type include liposomal derived systems, poly-lysine conjugates, and artificial viral envelopes.

In particular embodiments, plasmid vectors are used in the practice of the present invention. Naked plasmids can be introduced into muscle cells by injection into the tissue. Expression can extend over many months, although the number of positive cells is typically low (Wolff et al., (1989) Science 247:247). Cationic lipids have been demonstrated to aid in introduction of nucleic acids into some cells in culture (Felgner and Ringold, (1989) Nature 337:387). Injection of cationic lipid plasmid DNA complexes into the circulation of mice has been shown to result in expression of the DNA in lung (Brigham et al., (1989) Am. J. Med. Sci. 298:278). One advantage of plasmid DNA is that it can be introduced into non-replicating cells.

In a representative embodiment, a nucleic acid molecule (e.g., a plasmid) can be entrapped in a lipid particle bearing positive charges on its surface and, optionally, tagged with antibodies against cell surface antigens of the target tissue (Mizuno et al., (1992) No Shinkei Geka 20:547; PCT publication WO 91/06309; Japanese patent application 1047381; and European patent publication EP-A-43075).

Liposomes that consist of amphiphilic cationic molecules are useful non-viral vectors for nucleic acid delivery in vitro and in vivo (reviewed in Crystal, Science 270: 404-410 (1995); Blaese et al., Cancer Gene Ther. 2: 291-297 (1995); Behr et al., Bioconjugate Chem. 5: 382-389 (1994); Remy et al., Bioconjugate Chem. 5: 647-654 (1994); and Gao et al., Gene Therapy 2: 710-722 (1995)). The positively charged liposomes are believed to complex with negatively charged nucleic acids via electrostatic interactions to form lipid:nucleic acid complexes. The lipid:nucleic acid complexes have several advantages as nucleic acid transfer vectors. Unlike viral vectors, the lipid:nucleic acid complexes can be used to transfer expression cassettes of essentially unlimited size. Since the complexes lack proteins, they can evoke fewer immunogenic and inflammatory responses. Moreover, they cannot replicate or recombine to form an infectious agent and have low integration frequency. A number of publications have demonstrated that amphiphilic cationic lipids can mediate nucleic acid delivery in vivo and in vitro (Feigner et al., Proc. Natl. Acad. Sci. USA 84: 7413-17 (1987); Loeffler et al., Methods in Enzymology 217: 599-618 (1993); Feigner et al., J. Biol. Chem. 269: 2550-2561 (1994)).

Several groups have reported the use of amphiphilic cationic lipid:nucleic acid complexes for in vivo transfection both in animals and in humans (reviewed in Gao et al., Gene Therapy 2: 710-722 (1995); Zhu et al., Science 261: 209-211 (1993); and Thierry et al., Proc. Natl. Acad. Sci. USA 92: 9742-9746 (1995)). U.S. Pat. No. 6,410,049 describes a method of preparing cationic lipid:nucleic acid complexes that have a prolonged shelf life.

VII. Subjects, Pharmaceutical Formulations, Dosages and Modes of Administration.

The present invention finds use in veterinary and medical applications. Suitable subjects include both avians and mammals, with mammals being preferred. The term “avian” as used herein includes, but is not limited to, chickens, ducks, geese, quail, turkeys and pheasants. The term “mammal” as used herein includes, but is not limited to, humans, bovines, ovines, caprines, equines, felines, canines, lagomorphs, etc. In particular embodiments, the subject is a human subject that has been diagnosed with or is considered at risk for diabetes mellitus (type I or type 11), is obese and/or has insulin resistance. Human subjects include neonates, infants, juveniles, and adults. In other embodiments, the subject is an animal model of diabetes, obesity or insulin resistance.

As one particular aspect, the invention provides a pharmaceutical formulation comprising a compound or pharmaceutically acceptable salt thereof that reduces ketogenic enzyme activity or a compound that enhances ketolytic or lipid oxidizing activity in a pharmaceutically acceptable carrier. As another aspect, the present invention provides a pharmaceutical formulation comprising a compound identified according to the screening methods of this invention or a pharmaceutically acceptable salt thereof in a pharmaceutically acceptable carrier.

In other particular embodiments, the present invention provides a pharmaceutical composition comprising an inhibitory oligonucleotide or delivery vector of the invention in a pharmaceutically-acceptable carrier.

By “pharmaceutically acceptable” it is meant a material that is not biologically or otherwise undesirable, i.e., the material can be administered to a subject without causing any undesirable biological effects such as toxicity.

The term “pharmaceutically acceptable salts” refers to physiologically and pharmaceutically acceptable salts of the compounds of the invention, i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., (1977) “Pharmaceutical Salts,” J. of Pharma Sci. 66:1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from the respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids including, for example, with inorganic acids, such as hydrochloric acid, hydrobromic acid, sulfuric acid or phosphoric acid; with organic acids such as carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as naturally-occurring alpha-amino acids, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine and iodine.

The formulations of the invention can optionally comprise medicinal agents, pharmaceutical agents, carriers, adjuvants, dispersing agents, diluents, and the like.

The compositions (e.g., delivery vectors, oligonucleotides or compounds, including pharmaceutically acceptable salts thereof) of the invention can be formulated for administration in a pharmaceutical carrier in accordance with known techniques. See, e.g., Remington, The Science And Practice of Pharmacy (latest edition). In the manufacture of a pharmaceutical formulation according to the invention, the composition is typically admixed with, inter alia, an acceptable carrier. The carrier can be a solid or a liquid, or both, and is optionally formulated with the composition as a unit-dose formulation, for example, a tablet, which can contain from about 0.01 or about 0.5% to 95% or 99% by weight of the composition. One or more compositions can be incorporated in the formulations of the invention, which can be prepared by any of the well-known techniques of pharmacy.

In particular embodiments, the composition is administered to the subject in a therapeutically effective amount, as that term is defined herein. Dosages of pharmaceutically active compositions can be determined by methods known in the art, see, e.g., Remington's Pharmaceutical Sciences (Maack Publishing Co., Easton, Pa.). The therapeutically effective dosage of any specific composition will vary somewhat from composition to composition, and patient to patient, and will depend upon the condition of the patient and the route of delivery. As a general proposition, a dosage from about 0.1 to about 10, 20, 50, 75 or 100 mg/kg body weight will have therapeutic efficacy, with all weights being calculated based upon the weight of the active ingredient, including salts.

With particular respect to delivery vectors of the invention, dosages will depend upon the mode of administration, the severity of the disease or condition to be treated, the individual subject's condition, age and species of the subject, the particular vector, and the nucleic acid to be delivered, and can be determined in a routine manner. In particular embodiments, the vector is administered to the subject in a therapeutically effective amount, as that term is defined above.

Typically, with respect to viral vectors, at least about 10³ virus particles, at least about 10⁵ virus particles, at least about 10⁷ virus particles, at least about 10⁹ virus particles, at least about 10¹¹ virus particles, at least about 10¹² virus particles, or at least about 10¹³ virus particles are administered to the subject per treatment. Exemplary doses are virus titers of about 10⁷ to about 10¹⁵ particles, about 10⁷ to about 10¹⁴ particles, about 108 to about 1013 particles, about 10¹⁰ to about 10¹⁵ particles, about 10¹¹ to about 10¹⁵ particles, about 10¹² to about 10¹⁴ particles, or about 10¹² to about 10¹³ particles.

In particular embodiments of the invention, more than one administration (e.g., two, three, four, or more administrations) can be employed over a variety of time intervals (e.g., hours, days, weeks, months, years etc.) to achieve therapeutic effects.

The present invention further provides liposomal formulations of the compositions disclosed herein. The technology for forming liposomal suspensions is well known in the art. When the composition or salt thereof is an aqueous-soluble salt, using conventional liposome technology, the same can be incorporated into lipid vesicles. In such an instance, due to the water solubility of the composition or salt, the composition or salt will be substantially entrained within the hydrophilic center or core of the liposomes. The lipid layer employed can be of any conventional type and can either contain cholesterol or can be cholesterol-free. When the composition or salt of interest is water-insoluble, again employing conventional liposome formation technology, the salt can be substantially entrained within the hydrophobic lipid bilayer which forms the structure of the liposome. In either instance, the liposomes which are produced can be reduced in size, as through the use of standard sonication and homogenization techniques.

The liposomal formulations containing the inventive compounds can be lyophilized to produce a lyophilizate which can be reconstituted with a pharmaceutically acceptable carrier, such as water, to regenerate a liposomal suspension.

In the case of water-insoluble composition, a pharmaceutical formulation can be prepared containing the water-insoluble composition, such as for example, in an aqueous base emulsion. In such an instance, the formulation contains a sufficient amount of pharmaceutically acceptable emulsifying agent to emulsify the desired amount of the composition. Particularly useful emulsifying agents include phosphatidyl cholines and lecithin.

The formulations of the invention include those suitable for oral, rectal, topical, buccal (e.g., sub-lingual), vaginal, parenteral (e.g., subcutaneous, intramuscular including skeletal muscle, cardiac muscle, diaphragm muscle and/or smooth muscle, intradermal, intravenous, intraperitoneal), topical (i.e., both skin and mucosal surfaces, including airway surfaces), intranasal, transdermal, intraarticular, intrathecal and inhalation administration, administration to the liver, as well as direct organ injection (e.g., into the liver, into the skeletal muscle, into the brain for delivery to the central nervous system, into the pancreas, etc.).

For injection, the carrier will typically be a liquid, such as sterile pyrogen-free water, pyrogen-free phosphate-buffered saline solution, bacteriostatic water, or Cremophor EL[R] (BASF, Parsippany, N.J.). For other methods of administration, the carrier can be either solid or liquid.

For oral administration, the formulation can be administered in solid dosage forms, such as capsules, tablets, and powders, or in liquid dosage forms, such as elixirs, syrups, and suspensions. The composition can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the composition in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the composition, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit\dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect of the present invention, there is provided an injectable, stable, sterile composition of the invention, in a unit dosage form in a sealed container. The composition is provided in the form of a lyophilizate which is capable of being reconstituted with a suitable pharmaceutically acceptable carrier to form a liquid composition suitable for injection thereof into a subject. When the active ingredient or salt thereof is substantially water-insoluble, a sufficient amount of emulsifying agent which is pharmaceutically acceptable can be employed in sufficient quantity to emulsify the active ingredient or salt in an aqueous carrier. One such useful emulsifying agent is phosphatidyl choline.

Formulations suitable for rectal administration are preferably presented as unit dose suppositories. These can be prepared by admixing the composition with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Pharmaceutical Research 3 (6):318 (1986)) and typically take the form of an optionally buffered aqueous solution of the composition. Suitable formulations comprise citrate or bis\tris buffer (pH 6) or ethanol/water and contain from 0.1 to 0.2M of the composition.

The composition can alternatively be formulated for nasal administration or administered to the respiratory system (e.g., the lungs) of a subject by any suitable means, but is preferably administered by an aerosol suspension of respirable particles comprising the composition, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al. (1992) J. Pharmacol. Toxicol. Methods 27:143-159. Aerosols of liquid particles comprising the composition can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the composition can likewise be produced with any solid particulate medicament aerosol generator, by techniques known in the pharmaceutical art.

Alternatively, one can administer the compositions of the invention in a local rather than systemic manner, for example, in a depot, implantable device or sustained-release formulation (e.g., to be implanted in skeletal muscle).

In representative embodiments of the invention, the composition is administered to the skeletal muscle or liver (e.g., liver parenchyma).

Illustrative methods of administering a composition of the invention to the liver include administration by a route including but not limited to: intravenous administration, intraportal administration, intrabiliary administration, intra-arterial administration, or direct injection into the liver parenchyma.

Illustrative methods of administering a composition of the invention to the skeletal muscle include administration by a route including but not limited to: intravenous administration, intra-arterial administration, direct administration to skeletal muscle, for example, by direct injection or by an implantable device or depot.

The foregoing is illustrative of the present invention, and is not to be construed as limiting thereof. The invention is defined by the following claims, with equivalents of the claims to be included therein.

### EXAMPLE 1

### Materials and Methods

Recombinant Adenoviruses. AdCMV-MCDΔ5 recombinant adenovirus contains the human MCD cDNA modified to encode a fully active enzyme that is preferentially localized to the cytosolic compartment (Mulder, et al. (2001) J. Biol. Chem. 276:6479-84). Control AdCMV-MCDₘᵤₜ adenovirus contains the human MCD cDNA with an amino acid substitution (Leu³⁹⁸−>Pro) that renders the enzyme catalytically inactive (Mulder, et al. (2001) supra). These viruses were amplified and purified for injection into rats using well-established methods (Becker, et al. (1994) Methods Cell Biol. 43:161-89).

Animal Experiments. Male Wistar rats (75-100 grams; Charles River) were given free access to standard chow (SC, Harlan Teklad 7007; Harlan Teklad Laboratories, Winfield, Iowa) or to a high-fat diet (HF, Harlan Teklad TD96001). After being fed on SC or HF diets for 11 weeks, animals received a single dose (1.0×10¹² plaque-forming unit (pfu)/500 grams body weight) of AdCMV-MCDΔ5 or AdCMV-MCDₘᵤₜ adenoviruses by tail vein injection. Animals were then caged individually and continued on either the SC or HF diets, with daily monitoring of body weight and food consumption. Four days after virus injection, food was withdrawn for 18 hours prior to collection of a large blood sample by heart puncture of anesthetized animals. Tissues were collected and stored at −80° C.

Hepatocyte Studies. Hepatocytes were isolated from overnight fasted male Wistar rats (180-225 grams) and cultured using standard methods (Massague and Guinovart (1977) FEBS Lett. 82:317-20). Recombinant adenoviruses were added at a titer of 50 pfu/cell for 2 hours at 37° C. MCD activity and oxidation of 9,10-³H(N)-palmitate (NEN, Boston, Mass.) was measured 48 hours after viral treatment using well-established methods (Antinozzi, et al. (1998) J. Biol. Chem. 273:16146-54; Lee, et al. (1997) Diabetes 46:408413).

Measurement of Plasma Metabolic Variables. Blood samples were collected into EDTA-rinsed vials for analysis of plasma variables. Levels of plasma triglycerides, glycerol, β-hydroxybutyrate and aspartate aminotransferase were measured using commercial kits (SIGMA Diagnostics, St. Louis, Mo.). Plasma-free fatty acids (FFA) were analyzed using a FFA half-micro test kit (Roche Diagnostics, Mannheim, Germany). Plasma insulin and leptin were analyzed by radioimmunoassay (Linco, St. Charles, Mo.). Plasma glucose was measured using a B-Glucose Analyzer (HemoCue, Sweden). Animals with plasma levels of aspartate-aminotransferase higher than 200 U/L were excluded due to potential liver damage.

Analysis of Insulin/AKT Signaling Pathway in Skeletal Muscle. Acute insulin stimulation was performed by intraportal injection of 10 U/kg body weight of fast-acting insulin (HUMULIN® R; Eli Lilly and Co., Indianapolis, Ind.) into anesthetized, overnight fasted rats. Immediately prior to and 8 minutes after insulin injection, the gastrocnemius muscle of each leg was clamp-frozen and processed according to a well-known method (Shao, et al. (2000) J. Endocrinol. 167:107-15). The supernatant fractions of muscle extracts (100 μg of protein) were resolved on 10% Tris-HCl CRITERION™ gels (BIO-RAD®, Hercules, Calif.) and transferred to SEQUI-BLOT™ PVDF membranes (BIO-RAD®). The blots were incubated overnight at 4° C. with anti-AKT-1, anti-phospho (Ser⁴⁷³)-AKT-1, anti-phospho (Ser⁹)-GSK-3β (New England Biolabs, Beverly, Mass.), or anti-AKT-2 (Summers, et al. (1999) J. Biol. Chem. 274:23858-23867) antibodies. Bands were detected using HRP-conjugated secondary antibody and the ECL™ Western Blot Analysis System (Amersham Biosciences, Piscataway, N.J.).

Tissue Triglyceride and LC Acetyl CoA Assays. Triglyceride content of liver, mixed gastrocnemius, soleus, or extensor digitorum longus muscle was measured using the Infinity Triglyceride Reagent (SIGMA, St. Louis, Mo.) (Milburn, et al. (1995) J. Biol. Chem. 270:1295-9; Muoio, et al. (1999) Am. J. Physiol. 276:E913-21). Individual and total long chain acyl CoA species were measured by LC/MS/MS (Yu, et al. (2002) J. Biol. Chem. 277:50230-6).

Real-Time Quantitative PCR (RTQ-PCR). Total RNA was prepared using the TRIzol reagent, treated with DNase I, and quantified using the RIBOGREEN® RNA quantitation kit (Molecular Probes, Eugene, Oreg.). RTQ-PCR was performed using an ABI PRISM 7000 Sequence Detection System instrument and software (PE Applied Biosystems, Inc., Foster City, Calif.). Primer/probe sets were designed using the manufactures software and sequences available in GENBANK.

Isolation of Mitochondria. Mitochondria were isolated from white and red gastrocnemius muscles. Muscles were excised and immediately placed in ice-cold modified Chapell-Perry buffer (100 mM KCL, 40 mM Tris-HCl, 10 mM Tris-Base, 5 mM MgSO₄, 1 mM EDTA, 1 mM ATP, pH 7.5) and separated into red, white, or mixed gastrocnemius; only red (RG) and white (WG) gastrocnemius were used in experiments herein. Muscles were placed into 2.0 mL (RG) or 4 mL (WG) of Chapell-Perry buffer. Samples were minced thoroughly on ice, diluted 10-fold (w/v) with Chapell-Perry buffer and then homogenized 2×15 seconds using an Ultra-Turrax at approximately 9,500 rpm. Homogenates were centrifuged at 650×g for 10 minutes at 4° C. and the supernatant was gravity filtered through four layers of surgical gauze and centrifuged at 8,500×g for 10 minutes at 4° C. Reactions were initiated by adding 40 μL isolated mitochondria to 160 μL of the incubation buffer (pH 7.4), yielding final concentrations of 100 mM sucrose, 10 mM Tris-HCl, 5 mM potassium phosphate, 80 mM potassium chloride, 1 mM magnesium chloride, 2 mM L-carnitine, 0.1 mM malate, 2 mM ATP, 0.05 mM co-enzyme A, 1 mM dithiothreitol, 0.2 mM EDTA and 0.5% bovine serum albumin, plus ¹⁴C- or ¹³C-labeled substrates. After incubating 60 minutes at 30° C., reactions were terminated by adding 100 μL 70% perchloric acid and substrate metabolism was determine using standard methods (Kim, et al. (2002) Am. J. Physiol. Endocrinol. Metab. 282:E1014-E1022). Proteins amounts were determined by the BCA method.

Isolation of Muscle Preparations. Soleus muscles were removed under anesthesia (sodium pentobarbital 25-35 mg/kg body weight), cleaned free of adipose and connective tissue, and carefully dissected into longitudinal strips from tendon to tendon using a 27-gage needle. Two strips from each muscle with an approximate mass of 25 mg (wet mass) was clamped in lucite clips to maintain consistent resting muscle length and tension throughout the preparation (Hulver, et al. (2003) Am. J. Physiol. Endocrinol. Metab. 284(4):E741-E747). Clipped muscle strips were placed in 3.0 mL of warmed (30° C) Krebs-Ringer buffer (low calcium Kreb's Henseleit bicarbonate buffer) containing 5.0 mM glucose and gassed with 95% O₂-5% CO₂ (pH 7.4) containing 4% bovine serum albumin. After a 30-minute preincubation period, muscle strips were incubated for 1-4 hours at 30° C. in the same incubation medium but with the addition of appropriate radiolabeled substrates (Muoio, et al. (1999) Am. J. Physiol. 276:E913-E921).

Cell culture. Early passages of rat L6 myoblasts (ATCC CRL-1458) from the American Tissue Culture Collection (Rockville, Md.) were grown in Dulbecco's Modified Eagle Medium (DMEM) with 10% Fetal Bovine Serum, 4.0 mM glutamine, and 50 mg/mL gentamycin, in a humidified incubator at 37° C., 5% CO₂. Myoblasts were grown on 100 mm dishes to 50-60% confluence and then subcultured onto 6 and 24-well collagen-coated plates for experiments. When cells were 70% confluent, they were induced to differentiate into myotubes by changing to low-serum DFM (DMEM, 2% horse-serum, 4.0 mM glutamine, 50 mg/ml gentamycin). By day 6, cells were fully confluent, differentiated into multinucleated, contracting myotubes (Muoio, et al. (2002) J. Biol. Chem. 277(29):26089-26097; Muoio, et al. (2002) Diabetes 51:901-909).

Substrate Metabolism. Substrate oxidation rates (fatty acid, glucose, ketone, leucine) in isolated muscle and cultured myocytes were assayed using standard methods (Muoio, et al. (2002) Diabetes 51:901-909; Muoio, et al. (1999) Am. J. Physiol. 276:E913-E921). Briefly, the production of [¹⁴C]-labeled [¹⁴C]CO₂ (complete oxidation) was measured and where appropriate, [¹⁴C]-labeled ASM, a measure of TCA cycle intermediates and acylcarnitine esters and ketones (incomplete oxidation) was measured, using a modified 48-well microtiter plate (Kim, et al. (2002) Am. J. Physiol. Endocrinol. Metab. 282:E1014-El022). [UL-¹⁴C]glucose incorporation into muscle glycogen was assayed by dissolving samples in 4 M KOH followed by precipitation at −20° C. (Muoio, et al. (1999) Biochem. J. 338:783-791). Radioactivity was determined by scintillation counting. Cell or tissue lysates from experiment using [U-¹³C]oleate, glucose or leucine were prepared for MS/MS acylcarnitine analysis as described herein. Rates of ketone production were determined by treating media and tissue lysates with sodium borodeuteride, thereby producing ketoacids labeled with deuterium. Trimethylsilyated extracts spiked with internal standards were analyzed by GC/MS, permitting determination of βHB and AcAc in a single analysis.

Acylcarnitine analyses. Stable-isotope-labeled acylcarnitines were used as internal standards. To 50 μL tissue homogenates, acetyl-[2H³_methyl]carnitine, propionyl-[2H³-methyl]carnitine, butyryl-[2H³-methyl]carnitine, octanoyl-[2H³-methyl]carnitine, and palmitoyl-[2H³-methyl]carnitine were added at 5, 1, 1, 1, and 2 mmol/L, respectively. Proteins were then precipitated by addition of 800 μL ethanol, and the supernatant was extracted twice with 800 μL of hexane to remove interfering lipids. The aqueous layer was transferred to a vial, dried down under a stream of dry nitrogen, then incubated with 100 μL of 3 mol/L HCl in methanol at 50° C. for 15 minutes. The derivatized sample was dried under nitrogen, and reconstituted with 100 μL of a methanol-glycerol (1:1, v/v) matrix containing 0.5% (w/v) octyl sodium sulfate. Aliquots of extracts were transferred to 96-well microtiter plates, which were sealed with a thin sheet of aluminum foil to limit solvent evaporation before analysis. Samples were injected directly into the electrospray ion source of a tandem mass spectrometer (QUATTRO-LC; Waters-MICROMASS®, Milford, Mass.) equipped with a Hewlett-Packard HP1100 LC pump and Gilson model 215 sample handler fitted with the Gilson 701H microtiter plate rack. Acylcarnitines were quantified using a signal intensity ratio to the closest internal standard, and related to concentrations using the slope derived from standard curves (Cox, et al. (2001) Hum. Mol. Genet. 10(19):2069-2077).

Analysis of Acylcarnitines in Liver Tissue. Specimens of tissue were homogenized in de-ionized water. The mixture was centrifuged to remove cellular debris and the supernate removed for analysis of acylcarnitines. To 50 μL homogenate were added 2.5 μL of a mixture of internal standards containing 0.1 nmol/μL d₃-acetylcarnitine+0.02 nmol/μL d₃-propionylcarnitine+0.02 nmol/μL d₃-butyrylcarnitine+0.02 nmol/μL d₃-octanoylcarnitine+0.04 nmol/μL d₃-palmitoylcarnitine. After vortex-mixing for 30 seconds, acetonitrile (400 μL) was added to de-proteinize, and the mixture was again vortex-mixed and centrifuged (2,000×g for 5 minutes). An aliquot of the supernate (200 μL) was transferred to a predetermined position in a 96-well plate (Evergreen, Los Angeles, Calif.). After all specimens to be analyzed were transferred to the plate, the solvent was evaporated under nitrogen at 50° C. for 20 minutes using a drying apparatus (SPE Dry-96; Jones Chromatography, Hengoed, UK). Residues were incubated with either 3 M MeOH-HCl at 50° C. for 15 minutes (Supelco Inc., Bellefonte, Pa.) or 3 M BuOH-HCl at 65° C. for 15 minutes (Regis Chemical Company, Morton Grove, Ill.), depending on whether methyl ester or butyl ester derivatives of the acylcarnitines were to be prepared. The reagent was evaporated under nitrogen (50° C. for 20 minutes) and 200 μL methanol:water (85:15, volume:volume) were added to each well. The plate was covered with aluminum foil to minimize evaporation, then placed on the autosampler for direct analysis by tandem mass spectrometry. Specimens were analyzed for acylcarnitines by direct injection electrospray tandem mass spectrometry according to standard methods using a QUATTRO MICRO™ LC-MS system (Waters-MICROMASS®, Milford, Mass.) equipped with a model HTS-PAL autosampler (Leap Technologies, Carrboro, N.C.) and a model 1100 HPLC solvent delivery system (Agilent Technologies, Palo Alto, Calif.) and a datasystem running MASSLYNX™ software. Acylcarnitine profiles were generated using a precursor scan function (m/z 99 for methyl esters or m/z 85 for butyl esters) and the concentration of each analyte determined from the ratio of that signal to its assigned internal standard. For several analytes, the lack of available analytical standards required reporting a dimensionless value based on the analyte to internal standard ratio.

### EXAMPLE 2

### Hepatic Expression of Malonyl CoA Decarboxylase Reverses Whole-Animal and Muscle Insulin Resistance in Rats with Diet-Induced Obesity Hepatocyte Studies.

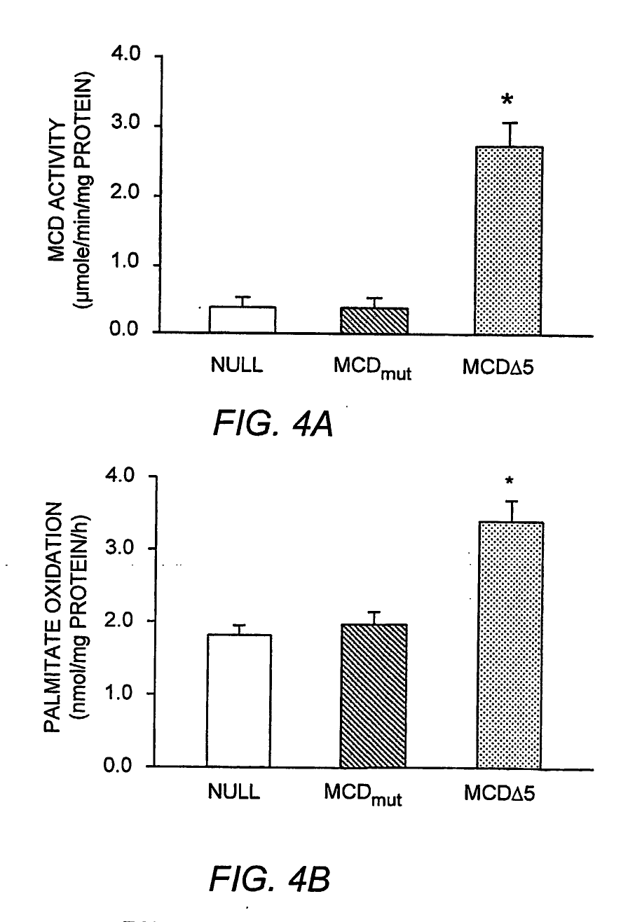

It has previously been demonstrated that treatment of insulinoma cells with a virus containing a modified MCD cDNA (AdCMV-MCDΔ5) lowers malonyl CoA levels and activates fatty acid oxidation (Mulder, et al. (2001) supra). Studies of similar design are provided herein, wherein hepatocytes were cultured in 25 mM glucose, a condition expected to elevate malonyl CoA levels and suppress fatty acid oxidation. Treatment of hepatocytes with AdCMV-MCDΔ5 raised MCD enzyme activity by 7-fold relative to control cells: that were either untreated or treated with a virus encoding a catalytically inactive form of MCD (AdCMV-MCDₘᵤₜ) (FIG. 4A). Fatty acid oxidation was increased by 86% and 71% in AdCMV-MCDΔ5-treated cells relative to the untreated and AdCMV-MCDmut-treated control groups, respectively (FIG. 4B).

Overexpression of MCD in Liver.

Recombinant adenoviruses delivered by intravenous injection in rats (Trinh, et al. (1998) J. Biol. Chem. 273:31615-20; O'Doherty, et al. (1999) Diabetes 48:2022-7; Gasa, et al. (2002) J. Biol. Chem. 277:1524-30) or mice (Herz and Gerard (1993) Proc. Natl. Acad. Sci. U.S.A. 90:2812-6) cause transgene expression preferentially in liver, with very low levels of expression in lung and no detectable expression in other extrahepatic tissues such as muscle, adipose, brain, and kidney. In the studies described herein, either AdCMV-MCDΔ5 or AdCMV-MCDₘᵤₜ, as a control, were infused into rats fed on standard chow (SC) or a high-fat diet (HF) for 11 weeks for analysis five days later. Relative to treatment with the control virus, AdCMV-MCDΔ5 infusion increased hepatic MCD enzyme activity by 2.7-fold and 2.3-fold in the SC and HF groups, respectively (Table 1).

TABLE 1

| | Standard Chow | | High-Fat Diet | |
|---|---|---|---|---|
| | MCDₘᵤₜ | MCDΔ5 | MCDₘᵤₜ | MCDΔ5 |
| | (n = 10) | (n = 10) | (n = 9) | (n = 11) |
| Glucose, mg/dL | 164 ± 10 | 161 ± 10 | 192 ± 12 | 176 ± 5 |
| FFA, mM | 0.34 ± 0.04 | 0.23 ± 0.03* | 0.56 ± 0.08^{#} | 0.36 ± 0.04* |
| TG, mg/dL | 55.63 ± 4.87 | 64.94 ± 4.97 | 98.62 ± 8.32^{#} | 117.58 ± 11.84 |
| Glycerol, mg/dL | 3.28 ± 0.51 | 2.73 ± 0.36 | 3.73 ± 0.36 | 3.41 ± 0.50 |
| βHB, mg/dL | 9.83 ± 0.94 | 9.83 ± 1.03 | 10.37 ± 2.45 | 9.43 ± 1.51 |
| Insulin, ng/dL | 1.60 ± 0.30 | 0.80 ± 0.23* | 4.26 ± 0.42^{#} | 1.72 ± 0.28* |
| Leptin, ng/dL | 1.76 ± 0.43 | 1.47 ± 0.17 | 30.27 ± 3.82^{#} | 20.67 ± 3.46 |
| Fat pad, gram | 6.1 ± 0.7 | 6.4 ± 0.5 | 14.6 ± 1.2^{#} | 16.4 ± 2.1 |
| Body Weight, gram | 426 ± 40 | 464 ± 57 | 539 ± 63^{#} | 514 ± 56 |
| MCD Activity, | 0.13 ± 0.01 | 0.32 ± 0.05* | 0.12 ± 0.01 | 0.28 ± 0.03* |
| μmol/min/mg | | | | |

Data are represented as mean ± S.E.;
*P < 0.05, compared with MCDₘᵤₜ-treated group fed with the same diet;
^{#}P < 0.05, compared with MCDₘᵤₜ-treated group fed on the SC diet.
MCDΔ5 = active virus;
MCDₘᵤₜ = inactive control.

Effects of Hepatic Overexpression of MCD on Metabolic Variables.

AdCMV-MCDΔ5 injection decreased insulin and free fatty acid (FFA) levels in SC rats, but otherwise had no significant effects on serum metabolites or hormones relative to AdCMV-MCDₘᵤₜ-injected controls (Table 1). Feeding of the HF diet caused clear metabolic derangements relative to SC feeding, similar to previous studies (Buettner, et al. (2000) Am. J. Physiol. Endocrinol. Metab. 278:E563-9; Gasa, et al. (2002) J. Biol. Chem. 277:1524-30). AdCMV-MCDₘᵤₜ-treated HF animals exhibited a 27% increase in body weight, a 2.4-fold increase in abdominal fat pad weight, a 65% increase in circulating FFA and a 77% increase in circulating triglycerides (TG) relative to AdCMV-MCDₘᵤₜ-treated SC controls (Table 1). The 2.6-fold increase in circulating insulin levels, coupled with a modest rise in glucose levels (17%, not statistically significant) was consistent with the presence of insulin resistance in the HF animals. Consistent with an increase in fat mass, leptin levels were increased by 17-fold in the HF versus SC groups.

Remarkably, several of the key metabolic perturbations induced by HF feeding were reversed by administration of AdCMV-MCDΔ5 (Table 1). Most prominent among these was the return of insulin levels to those of the SC controls, indicating amelioration of insulin resistance, and a return of FFA levels to normal, whereas TG levels remained elevated. Body weight, abdominal fat pad mass (Table 1) and food consumption were not different between AdCMV-MCDΔ5- and AdCMV-MCDₘᵤₜ-treated HF rats. These data show the development of a metabolic syndrome resembling early stage type 2 diabetes in HF rats, and also document the reversal of several key metabolic abnormalities in response to hepatic overexpression of MCD.

Hepatic Expression of MCD Relieves Insulin Resistance in Muscles.

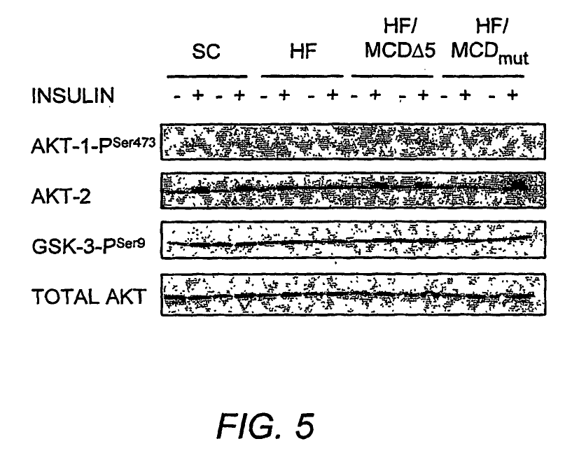

The regulation of key components of the known insulin signaling pathway in muscle was examined. Immunoblot analysis with an anti-phospho (Ser⁴⁷³) Akt1 antibody or with an antibody that detects both phosphorylated and unphosphorylated forms of Akt2 (Summers, et al. (1999) J. Biol. Chem. 274:23858-23867) showed that acute insulin-mediated phosphorylation of Akt proteins was clearly impaired in animals fed on the HF diet. Hepatic overexpression of MCD restored the robust insulin-mediated phosphorylation of both forms of Akt, whereas delivery of the AdCMV-MCDₘᵤₜ virus caused little or no improvement (FIG. 5). Similar findings were obtained with an anti-phospho (Ser⁹) GSK-3β antibody. Taken together, these data strongly indicate that expression of MCD in liver of HF rats results in amelioration of muscle insulin resistance, as assessed by both metabolic and cell signaling assays.

Effects of Hepatic Overexpression of MCD on Liver and Muscle Triglyceride Levels.

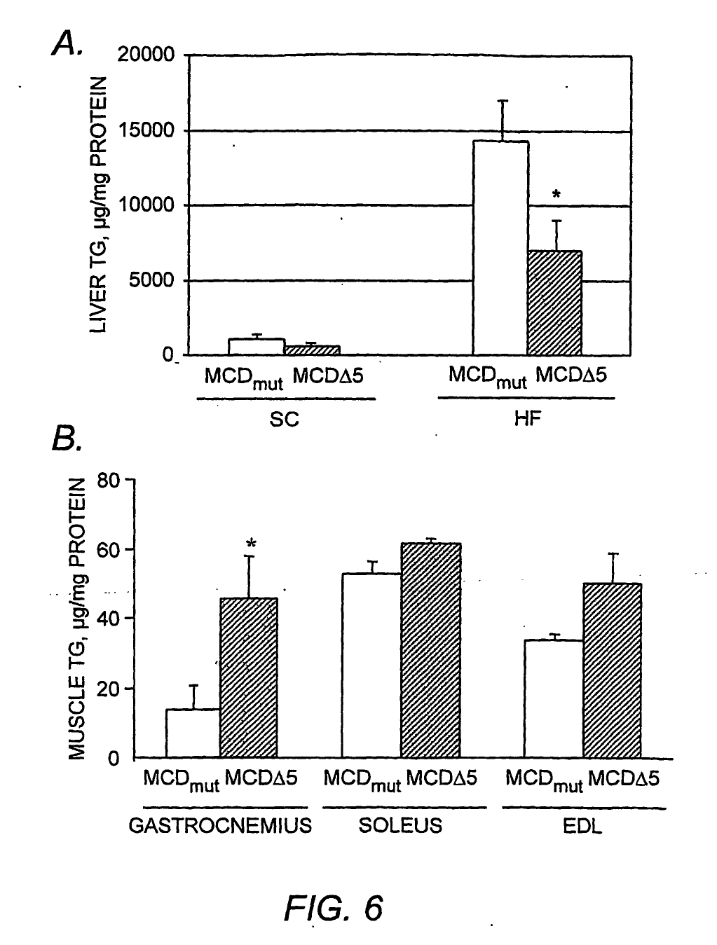

To investigate the role of lipid accumulation in these studies, TG content was measured in liver and in mixed gastrocnemius, soleus, and extensor digitorum longus muscles, which have distinct fiber type compositions. AdCMV-MCDₘᵤₜ-treated rats fed on the HF diet had more than 10 times as much TG in liver as SC controls, and these levels were reduced by 60% in AdCMV-MCDΔ5-treated HF animals (FIG. 6, panel A).

Unexpectedly, treatment of HF rats with AdCMV-MCDΔ5 increased the TG content of gastrocnemius muscle 3-fold (FIG. 6, panel B), relative to AdCMV-MCDₘᵤₜ-treated HF controls. A similar trend toward increased TG was observed in soleus and extensor digitorum longus muscles from the MCD overexpressing HF rats (FIG. 6, panel B). Thus, in the model described herein, whole animal and muscle insulin sensitivity is enhanced by a mechanism that is independent of lowering of muscle lipid content.

### EXAMPLE 3

### Diet-Induced Changes in Muscle Insulin Sensitivity Correlate with Muscle βOH-Butyrate (βHB) Levels

Fatty acids are activated for metabolic processing by esterification with coenzyme A (CoA) through a thioester bond. This process renders the metabolite impermeable to cellular membranes, thus effectively separating acyl-CoA esters into several physically and functionally distinct pools within various subcellular compartments. The carnitine acyltransferases represent a family of enzymes that are localized in various subcellular organelles and catalyze the formation of short, medium and long-chain acyl-carnitines, and in exchange regenerate free CoA (R—CO—S—CoA+carnitine-OH═RCO—O−carnitine+CoA—SH) (Zammit (1999) Prog. Lipid Res. 38(3):199-224). These reactions provide a mechanism whereby the cell can modulate the acyl- and acetyl-CoA/CoA ratios within subcellular compartments while also enabling the transfer of acyl moieties between these compartments. Thus, acylcarnitine profiles obtained from cell lysates or whole tissue samples provide a composite representation of acyl-CoA metabolism occurring within various subcellular organelles (mostly mitochondria) and are frequently used to evaluate physiological and pathophysiological changes in fatty acid homeostasis (Cox, et al. (2001) Hum. Mol. Genet. 10(19):2069-2077; Shen, et al. (2000) J. Inherit. Metab. Dis. 23(1):27-44; Matern, et al. (1999) Pediatr. Res. 46(1):4549; Van Hove, et al. (1993) Am. J. Hum. Genet. 52(5):958-966).

Accordingly, using tandem MS-based acylcarnitine profiling (Millington, et al. (1990) J. Inherit. Metab. Dis. 13(3):321-324), profiles of acylcarnitine intermediates were obtained in muscle samples from rats exposed to manipulations designed to induce or ameliorate insulin resistance. Normal Wistar rats were allowed to feed ad libitum either on SC or a HF diet for a period of 10 weeks. Rats fed on the HF diet developed severe insulin resistance. Subsets of animals from the HF and SC-fed groups were studied either in the ad libitum fed state or following an 18-hour period of starvation. Additionally, acylcarnitine intermediates were profiled in muscle samples from rats fed the HF diet and then treated with the MCD adenovirus or an inactive control virus (as described above).

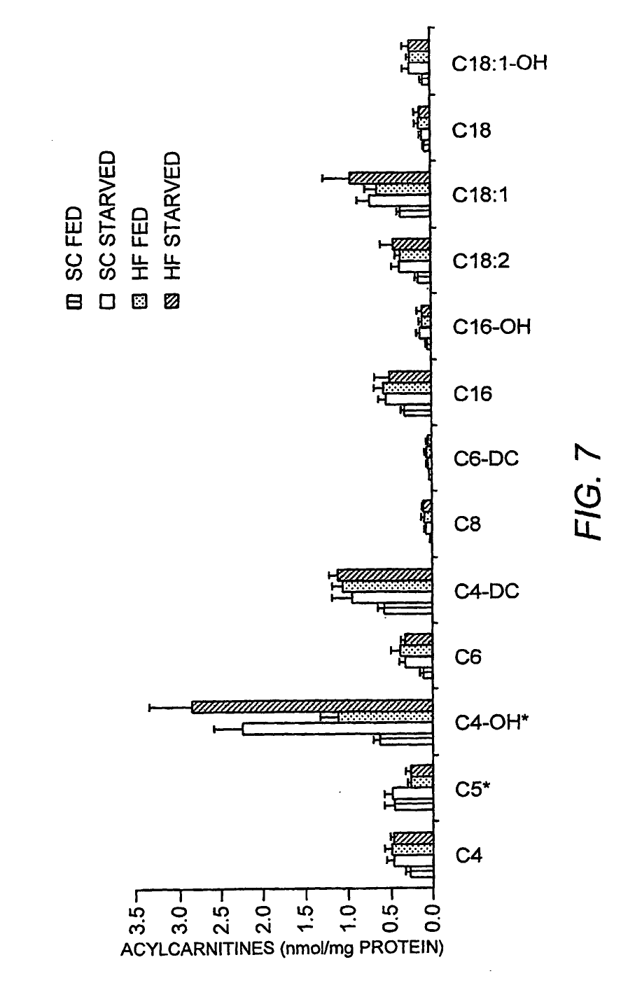

In the SC group, starvation increased several medium and long-chain acyl-carnitine intermediates, consistent with increased fatty acid delivery and high rates of β-oxidation that occur in the fasted condition. In muscles from HF-fed animals most acylcarnitine intermediates were persistently elevated (compared to the SC-fed group) but did not change during the fed-to-starved transition. This pattern is consistent with the high serum non-esterified fatty acid levels that were measured in HF fed group (Table 1). Most notably, starvation, which represents a state of transient insulin resistance, produced a dramatic increase in the βHB-carnitine derivative (C4-OH), reflecting muscle accumulation of this ketone. Similarly, in insulin resistant muscles from the HF-fed rats, βHB-carnitine levels were increased 75% over the SC-fed controls. This intermediate was of particular interest because it was the most abundant carnitine derivative among the C4-C20 chain lengths and because it was the only intermediate that increased with starvation in the HF group. Thus, βHB is the only lipid that increases in response to all of the maneuvers known to cause insulin resistance in this experiment. Another potentially important observation was that the HF diet decreased the isovaleryl-carnintine ester (C5), a leucine-derived ketogenic intermediate that is produced in the mitochondria (FIG. 7). Previous studies have shown that muscle possesses a marked ability to degrade leucine as an energy source, and moreover, that leucine catabolism is enhanced by high fatty acid (Shimomura, et al. (1990) J. Appl. Physiol. 68(1):161-165). Thus, decreased C5 levels in mitochondria may reflect increased use of this intermediate as a ketogenic substrate.

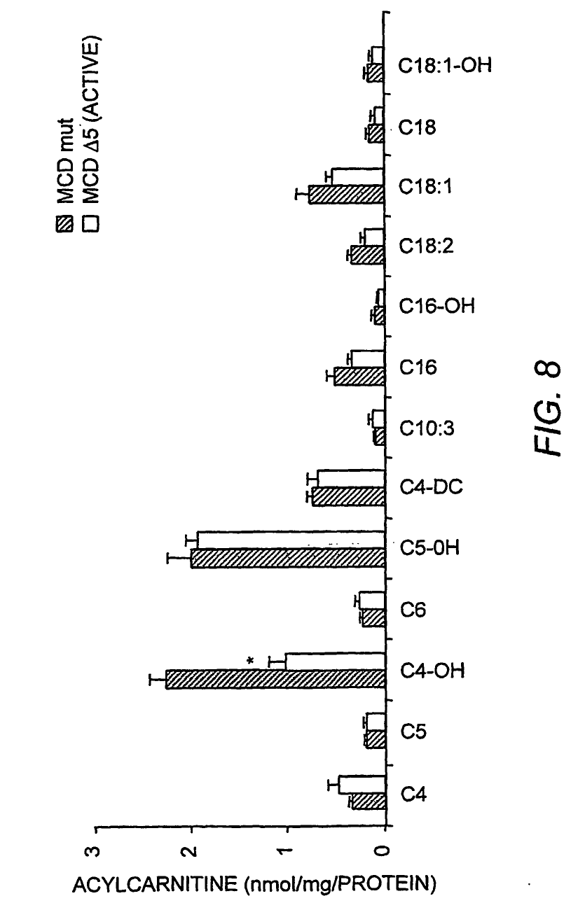

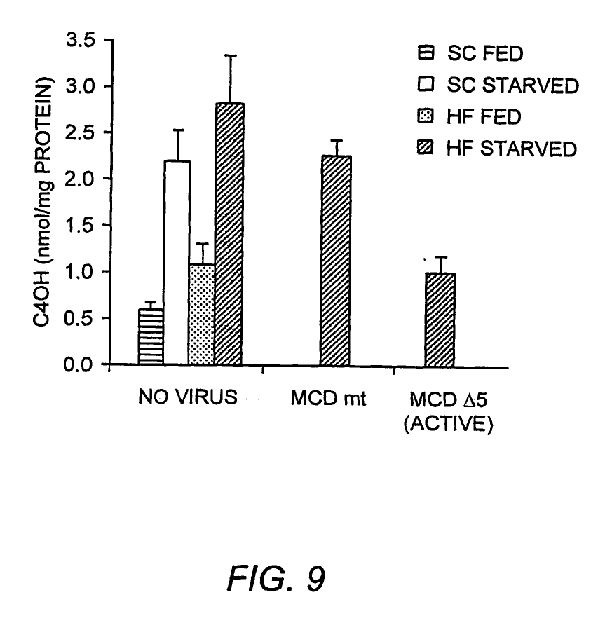

The most striking link between βHB and insulin resistance was obtained when acylcarnitine levels were examined in muscles from rats fed on the HF diet and treated with either the active MCD virus or the inactive control virus. Treatment with the active MCD virus, which restored insulin sensitivity, decreased muscle βHB levels by 55% (FIG. 8), with only marginal changes in all other short/medium acylcarnitine species. The MCD treatment also decreased several long-chain fatty acylcarnitines, but to a lesser extent (13-40%) than the βHB. The lower long-chain fatty acylcarnitine levels may reflect a decrease in their rate of formation due to diminished delivery of non-esterified fatty acids. This, in turn, would favor lower production of βHB, since the end products of p-oxidation provide substrate for ketogenesis. Taken together, the changes in muscle βHB levels observed in both the fasting-feeding experiments and in the MCD virus treatment study (FIG. 9) establish a strong positive association between elevated muscle ketones and insulin desensitization. Thus, chronically elevated βHB levels in muscles from HF fed rats may play a causal role in their metabolic syndrome. Since serum ketones were not different between the MCD-treated vs. the control group, lower muscle βHB levels in the MCD-treated rats likely reflect decreased endogenous ketone production due to the lowering of serum non-esterified fatty acids, which would not only reduce substrate supply but might also relieve lipid-mediated induction of ketogenic enzymes (FIG. 1). Consistent with this premise, results from microarray studies showed that chronic exposure to a high fat diet increased muscle mRNA expression of several genes that promote fatty acid oxidation (Table 2). Moreover, the high fat diet also increased muscle expression of the ketogenic enzyme, mitochondrial HMG-CoA synthase (mHS2), thus implicating muscle as a potential source of

### TABLE2

### Select Fatty Acid Oxidation Genes Upregulated in Soleus Muscle from HF vs. SC

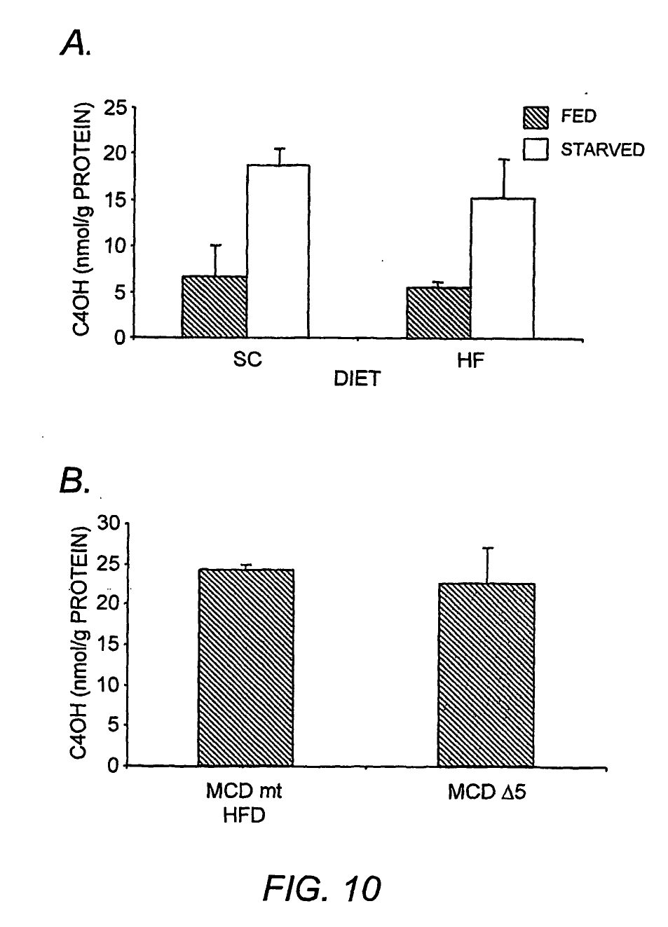

Common Genbank Map Function
Hmgcs2 M33648 2q34 ketogenesis 3-hydroxy-3-methylglutaryl-Coenzyme A synthase 2
Dci NM_{—}017306 10q12 FAO dodecenoyl-coenzyme A delta isomerase
Cpt1b NM_{—}013200 7q34 FAO carnitine palmitoyltransferase 1b
3kt NM_{—}130433 FAO acetyl-Coenzyme A acyltransferase 2 (mitochondrial 3-oxoacyl-Co A thiolase)
Eci D00729 FAO R. norvegicus mRNA for delta3, delta2-enoyl-CoA isomerase
Acacb AB004329 12q16 FAO acetyl-Coenzyme A carboxylase beta
Ucp3 NM_{—}013167 1q32 FAO uncoupling protein 3
Decr1 D00569 5ql3 FAO 2,4-dienoyl CoA reductase 1, mitochondrial
Hadhsc AF095449 2q42 FAO L-3-hydroxyacyl-Coenzyme A dehydrogenase, short chain
OxctVScot NM_{—}024188 15 A1 ketolysis 3-oxoacid CoA transferase
Ech1 NM_{—}022594 1q21 FAO enoyl coenzyme A hydratase 1
Pdk4 AF034577 4q13 FAO pyruvate dehydrogenate kinase 4 ketone metabolite. Finally, MCD treatment did not decrease liver βHB levels (FIG. 10) and similarly, did not change serum ketone concentrations. In the aggregate, these data indicate that the MCD-mediated decrease in muscle βHB was due to suppression of local (intramuscular) ketogenesis.

### EXAMPLE 4

### Insulin Resistance and Intramuscluar Ketogenesis

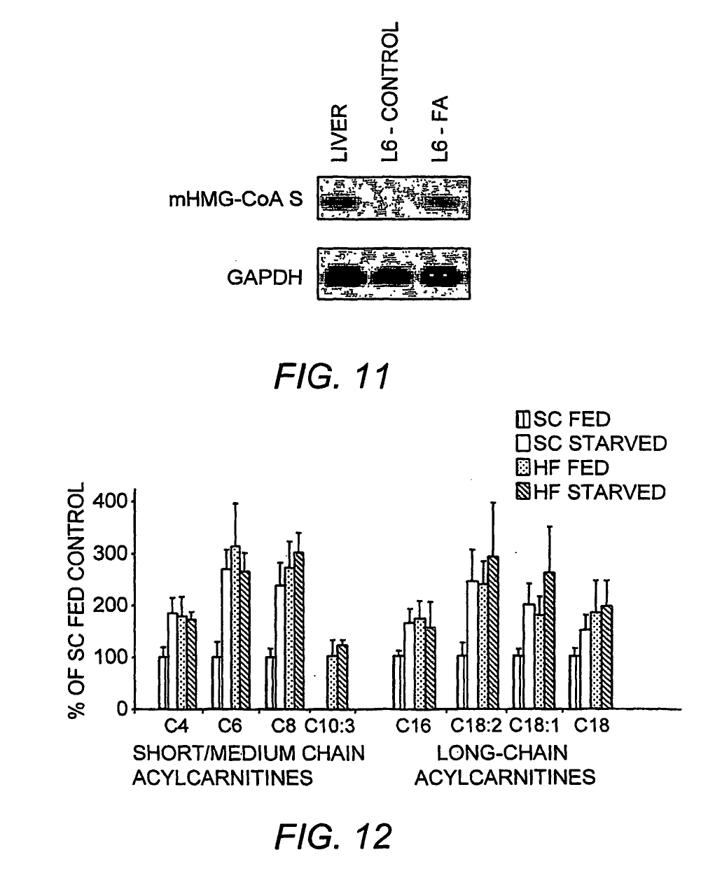

Collectively, it has been demonstrated that fatty acids can function as molecular regulators of muscle lipid metabolism and that their gene-regulatory properties are at least partly mediated by PPARs α and δ. Since these transcription factors (particularly PPARα) are also known to control hepatic ketogenesis, similar mechanisms of ketone regulation may be operative in skeletal muscle. Thus, HF feeding, which causes dyslipidemia and chronically elevated non-esterified fatty acids, may trigger PPAR-mediated expression of ketogenic genes in skeletal muscle, thereby contributing to increased synthesis and accumulation of βHB. To analyze this, it was determined whether mRNA expression of mitochondrial HMG-CoA synthase (mHS), a rate-controlling enzyme in hepatic ketogenesis, can be induced in rat L6 myotubes by exposure to high fatty acids. Gene expression levels were assayed by conventional RT-PCR (FIG. 11) and confirmed by real-time quantitative PCR (RTQ-PCR) using an ABI 7000 Detection System and mRNA from rat liver was used as a positive control. It was found that mHS mRNA levels were barely detectable in control myotubes that were maintained in the absence of fatty acid, however, when cells were incubated 24 hours in the presence of 500 μM oleate or a potent pharmacological PPARα activator, mHS expression increased dramatically. Using the same muscle cell culture system it was also found that 48 hours pre-incubation with 500 μM fatty acid inhibits both basal and insulin-stimulated [UL-¹⁴C]glucose oxidation and incorporation into glycogen. Thus, these results demonstrate that a connection exists between induction of mHS and impaired glucose handling in skeletal myocytes.

Several lines of evidence support the notion that a high FA supply leads to accelerated β-oxidation and intramuscular ketogenesis. In animals fed a standard chow (SC) diet, muscle levels of several medium and long-chain acyl-carnitine intermediates increase during the fed to starved transition (FIG. 12), in parallel with a rise in serum FFA. Likewise, when animals are fed a high fat (HF) diet, circulating lipids increase and most intramuscular acylcarnitine intermediates are persistently elevated. This pattern suggests that both starvation and chronic HF feeding increase FA uptake and metabolism by muscle mitochondria. Corroborating evidence also comes from transcriptional profiling analyses, which showed that several β-oxidative and ketogenic genes (including mitochondrial HMG-CoA synthase) were up-regulated in muscle from HF vs. SC rats (Table 2). Finally, results from studies using isolated mitochondria are consistent with the possibility that chronic fat exposure increases the ketogenic potential of muscle.

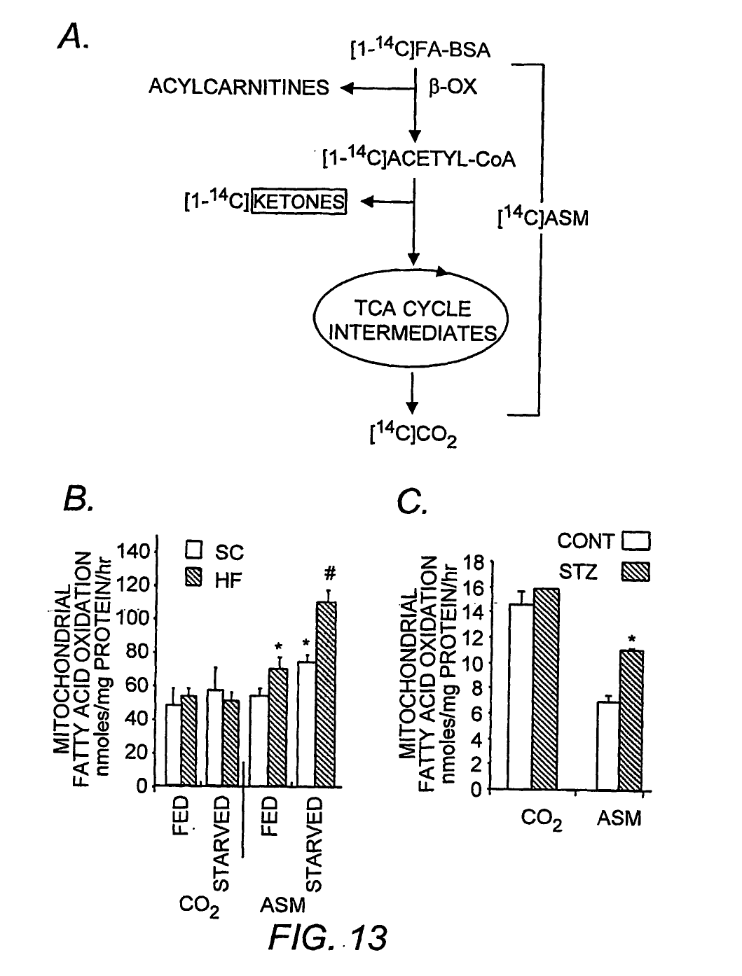

Mitochondrial [1-¹⁴C]oleate degradation to CO₂ and acid soluble metabolites (ASM) was measured to evaluate complete and incomplete FA oxidation, respectively (FIG. 13, Panel A). Rates of [¹⁴C]oleate oxidation to CO₂ were unchanged in mitochondria from HF compared to SC-fed rats; however, the HF diet increased production of [¹⁴C]ASM (FIG. 13, Panel B). Similar results were obtained when [¹⁴C]oleate oxidation in muscle mitochondria from STZ-treated rats was compared against those from controls (FIG. 13, Panel C). The acid-soluble fraction includes β-oxidative and TCA cycle intermediates as well as free ketones. Thus, increased label incorporation into ASM without a corresponding change in CO₂ could reflect a mismatch between β-oxidation and TCA flux that favors ketogenesis.

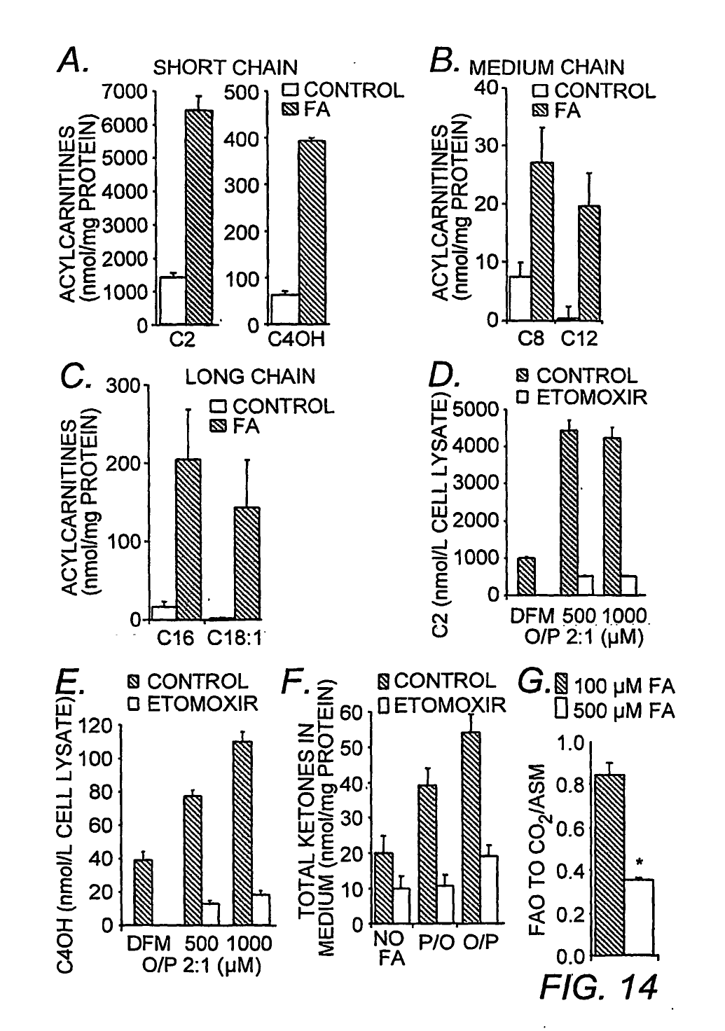

In studies that used L6 myocytes as a model, a similar lipid-induced phenotype has been observed, including accumulation of short, medium and long chain acylcarnitines (FIG. 14, Panels A-F), induction of β-oxidative and ketogenic genes (not shown), and increased rates of incomplete β-oxidation (FIG. 14, Panel G). Furthermore, it has been shown that exposure of cultured muscle cells to high FA causes intracellular accumulation of βHB-carnitine (C40H) as well as increased production of free ketones (FIG. 14, Panels D-F). Both accumulation of βHB and production of free ketones corresponded with excessive generation of the ketogenic precursor, acetyl-CoA (C2). Accumulation of all of these lipid metabolites was prevented by co-administration of etomoxir, which prevents FA transport into the mitochondria. These results imply that ketogenesis occurs when rates of β-oxidation exceed energy demand and, further, that conditions that favor accumulation of both acetyl units and reducing equivalents are likely to promote the generation of βHB, and might also represent a state of altered mitochondrial function and/or oxidative stress.

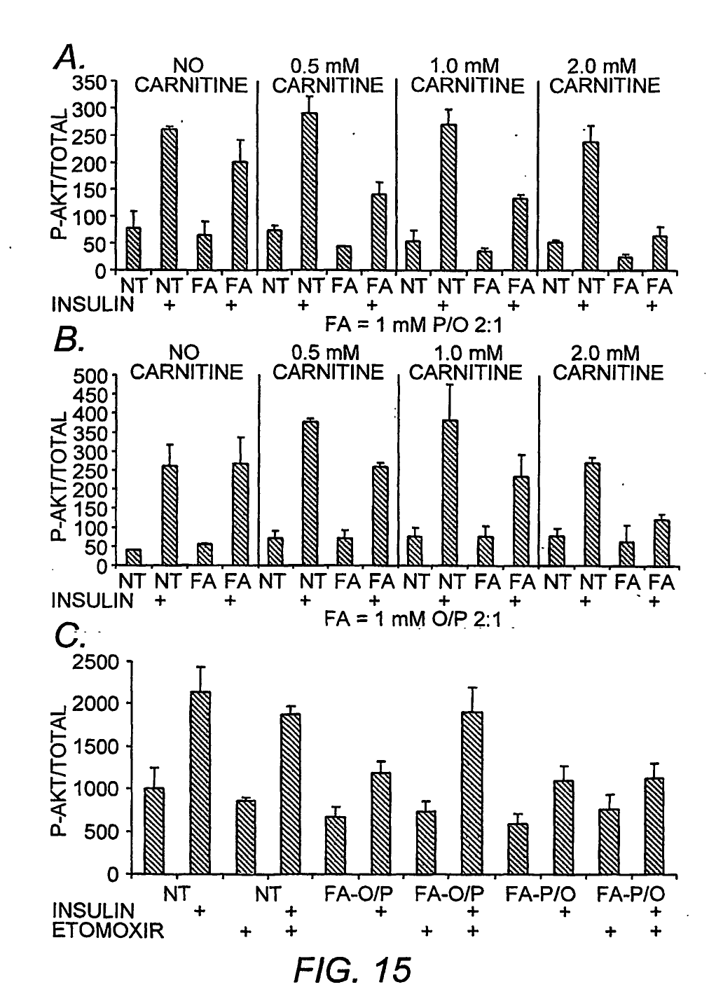

It has also been found that 24 h exposure of L6 myotubes to high FA impairs insulin signaling. The model predicts that the development of insulin resistance requires FA uptake into muscle mitochondria and subsequent β-oxidation. To test this model, signaling experiments were performed in which mitochondrial FA metabolism was inhibited by carnitine deficiency or etomoxir. The addition of carnitine exacerbated lipid-induced insulin resistance in a dose-dependent manner (FIG. 15, Panels A-B). Conversely, when etomoxir was present, the cells were at least partly protected against the lipid (FIG. 15, Panel C).

In the aggregate, these studies provide strong evidence that the accumulated βHB in muscle of insulin resistant rats was derived from intramuscular catabolism of FA, and support the hypothesis that lipid induced insulin resistance is mediated by a mitochondrial signal/metabolite that is at least closely associated with changes in muscle ketogenesis.

The foregoing examples are illustrative of the present invention, and are not to be construed as limiting thereof. The invention is described by the following claims, with equivalents of the claims to be included therein.

1. A method of treating diabetes comprising administering a compound that reduces skeletal muscle ketone levels to a diabetic subject in a therapeutically effective amount to reduce skeletal muscle ketone levels.

2. (canceled)

3. The method of claim 1, wherein the compound enhances ketolytic activity in skeletal muscle.

4. The method of claim 3, wherein the compound enhances the activity of a ketolytic enzyme in skeletal muscle.

5. The method of claim 1, wherein the compound reduces ketogenic activity in skeletal muscle.

6. The method of claim 5, wherein the compound reduces the activity of a ketogenic enzyme in skeletal muscle.

7. The method of claim 1, wherein the compound enhances hepatic fatty acid oxidation.

8. The method of claim 7, wherein the compound enhances the activity of a hepatic fatty acid oxidizing enzyme.

9. The method of claim 1, wherein the compound is a succinate ester or a succinate precursor.

10. The method of claim 1, wherein the compound is a polypeptide.

11. The method of claim 9, wherein the compound is an antibody.

12. The method of claim 1, wherein the compound is a nucleic acid molecule.

13-14. (canceled)

15. A delivery vector comprising a heterologous nucleic acid that encodes a ketolytic enzyme, wherein the heterologous nucleic acid is operably linked to a control element that directs the expression of the nucleic acid in skeletal muscle cells.

16. The delivery vector of claim 15, wherein the ketolytic enzyme is selected from the group consisting of acetoacetate:succinyl CoA:3oxoacid CoA transferase (SCOT) and α-ketoacid dehydrogenase.

17-18. (canceled)

19. A delivery vector comprising a heterologous nucleic acid that encodes an enzyme that mediates fatty acid oxidation, wherein the heterologous nucleic acid is operably linked to a control element that directs the expression of the nucleic acid in hepatic cells.

20. The delivery vector of claim 19, wherein the enzyme that mediates fatty acid oxidation is selected from the group consisting of malonyl CoA decarboxylase, carnitinepalmitoyltransferase I, carnitinepalmitoyltransferase II, carnitine acyltranslocase, acyl-CoA dehydrogenase, enoyl-CoA hydratase, 3-L-hydroxyacyl-CoA dehydrogenase, and β-ketoacyl-CoA thiolase.

21-22. (canceled)

23. An inhibitory oligonucleotide that is at least 8 nucleotides in length and specifically hybridizes to a target sequence encoding a ketogenic enzyme and reduces production of the ketogenic enzyme.

24. The inhibitory oligonucleotide of claim 23, wherein the ketogenic enzyme is selected from the group consisting of β-hydroxybutyrate dehydrogenase, mitochondrial HMG-CoA synthase, acetoacetyl-CoA thiolase, and HMG-CoA lyase.

25-31. (canceled)

32. A pharmaceutical formulation comprising the delivery vector of claims 15 in a pharmaceutically acceptable carrier.

33. A pharmaceutical formulation comprising the delivery vector of claim 19 in a pharmaceutically acceptable carrier.

34. A pharmaceutical formulation comprising the inhibitory oligonucleotide of claim 23 in a pharmaceutically acceptable carrier.

35. A method of reducing ketone levels in a skeletal muscle cell comprising contacting the skeletal muscle cell with a delivery vector according to claim 15 in an amount effective to reduce ketone levels in the skeletal muscle cell.

36-42. (canceled)

43. A method of treating diabetes comprising administering a delivery vector according to claim 15 to a diabetic subject in a therapeutically effective amount to reduce skeletal muscle ketone levels.

44-46. (canceled)

47. A method of reducing ketone levels in skeletal muscle comprising administering a delivery vector according to claim 19 to a subject in an amount effective to reduce skeletal muscle ketone levels.

48. A method of treating diabetes comprising administering a delivery vector according to claim 19 to a diabetic subject in a therapeutically effective amount to reduce skeletal muscle ketone levels.

49-51. (canceled)

52. A method of identifying a candidate compound for the treatment of diabetes, comprising
contacting a ketogenic enzyme with a compound; and
detecting binding of the compound to the ketogenic enzyme, wherein binding to the ketogenic enzyme identifies the compound as a candidate for the treatment of diabetes.

53. A method of identifying a candidate compound for the treatment of diabetes, comprising
contacting a ketogenic enzyme with a compound; and
detecting a reduction in ketogenic enzyme activity, wherein a reduction in ketogenic enzyme activity identifies the compound as a candidate for the treatment of diabetes.

54. A method of identifying a candidate compound for the treatment of diabetes, comprising:
contacting a cell that produces ketones with a compound;
detecting ketone levels in the cell, wherein a reduction in ketone levels identifies the compound as a candidate for the treatment of diabetes.

55. (canceled)

56. A method of identifying a candidate compound for the treatment of diabetes, comprising:
contacting a cell that produces a ketogenic enzyme with a compound;
detecting an indicia selected from the group consisting of:
(a) the concentration of the ketogenic enzyme, (b) the ketogenic enzyme activity, (c) the level of mRNA encoding the ketogenic enzyme, and (d) any combination of (a) to (c),
wherein a reduction in the level of the indicia in the cell identifies the compound as candidate for the treatment of diabetes.

57-58. (canceled)

59. A method of identifying a candidate compound for the treatment of diabetes, comprising:
administering a compound to a mammalian subject, detecting skeletal muscle ketone levels in the mammalian subject, wherein a reduction in skeletal muscle ketone levels identifies the compound as a candidate for the treatment of diabetes.

60. (canceled)

61. A method of identifying a candidate compound for the treatment of diabetes, comprising:
administering a compound to a mammalian subject,
detecting an indicia in skeletal muscle selected from the group consisting of:
(a) the concentration of a ketogenic enzyme,
(b) a ketogenic enzyme activity,
(c) mRNA encoding a ketogenic enzyme, and
(d) any combination of (a) to (c),
wherein a reduction in the level of the indicia in skeletal muscle identifies the compound as a candidate for the treatment of diabetes.

62-63. (canceled)

64. A method of identifying a candidate compound for the treatment of diabetes, comprising:
administering a compound to a transgenic non-human mammal that exhibits insulin resistance, the transgenic non-human mammal comprising an isolated nucleic acid encoding a ketogenic enzyme,
detecting the level of insulin resistance in the transgenic non-human mammal after administration of the compound, wherein a reduction in the level of insulin resistance identifies the compound as a candidate for the treatment of diabetes.

65. (canceled)

66. A method of identifying a candidate compound for the treatment of diabetes, comprising
contacting an enzyme that mediates fatty acid oxidation with a compound; and
detecting binding of the compound to the enzyme, wherein binding to the enzyme identifies the compound as a candidate for the treatment of diabetes.

67. A method of identifying a candidate compound for the treatment of diabetes, comprising
contacting an enzyme that mediates fatty acid oxidation with a compound; and
detecting an enhancement in enzyme activity, wherein an enhancement in enzyme activity identifies the compound as a candidate for the treatment of diabetes.

68. A method of identifying a candidate compound for the treatment of diabetes, comprising:
contacting a cell that produces an enzyme that mediates fatty acid oxidation with a compound;
detecting an indicia selected from the group consisting of:
(a) the concentration of the enzyme,
(b) the enzyme activity,
(c) the level of mRNA encoding the enzyme, and
(d) any combination of (a) to (c),
wherein an enhancement in the level of the indicia in the cell identifies the compound as candidate for the treatment of diabetes.

69-70. (canceled)

71. A method of identifying a candidate compound for the treatment of diabetes, comprising:
administering a compound to a mammalian subject,
detecting an indicia in skeletal muscle selected from the group consisting of:
(a) the concentration of an enzyme that mediates fatty acid oxidation,
(b) the activity of an enzyme that mediates fatty acid oxidation,
(c) mRNA encoding an enzyme that mediates fatty acid oxidation, and
(d) any combination of (a) to (c),
wherein an enhancement in the level of the indicia in skeletal muscle identifies the compound as a candidate for the treatment of diabetes.

72-73. (canceled)

74. A method of identifying a candidate compound for the treatment of diabetes, comprising:
administering a compound to a transgenic non-human mammal that exhibits insulin resistance, the transgenic non-human mammal comprising an isolated nucleic acid encoding an enzyme that mediates fatty acid oxidation,
detecting the level of insulin resistance in the transgenic non-human mammal after administration of the compound, wherein a reduction in the level of insulin resistance identifies the compound as a candidate for the treatment of diabetes.

75. (canceled)

76. A method of identifying a candidate compound for the treatment of diabetes, comprising
contacting a ketolytic enzyme with a compound; and
detecting binding of the compound to the ketolytic enzyme, wherein binding to the ketolytic enzyme identifies the compound as a candidate for the treatment of diabetes.

77. A method of identifying a candidate compound for the treatment of diabetes, comprising
contacting a ketolytic enzyme with a compound; and
detecting an enhancement in ketolytic enzyme activity, wherein an enhancement in ketolytic enzyme activity identifies the compound as a candidate for the treatment of diabetes.

78. A method of identifying a candidate compound for the treatment of diabetes, comprising:
contacting a cell that produces a ketolytic enzyme with a compound;
detecting an indicia selected from the group consisting of:
(a) the concentration of the ketolytic enzyme,
(b) the ketolytic enzyme activity,
(c) the level of mRNA encoding the ketolytic enzyme, and
(d) any combination of (a) to (c),
wherein an enhancement in the level of the indicia in the cell identifies the compound as candidate for the treatment of diabetes.

79-80. (canceled)

81. A method of identifying a candidate compound for the treatment of diabetes, comprising:
administering a compound to a mammalian subject,
detecting an indicia in skeletal muscle selected from the group consisting of:
(a) the concentration of a ketolytic enzyme,
(b) the activity of a ketolytic enzyme,
(c) mRNA encoding a ketolytic enzyme, and
(d) any combination of (a) to (c),
wherein an enhancement in the level of the indicia in skeletal muscle identifies the compound as a candidate for the treatment of diabetes.

82-83. (canceled)

84. A method of identifying a candidate compound for the treatment of diabetes, comprising:
administering a compound to a transgenic non-human mammal that exhibits insulin resistance, the transgenic non-human mammal comprising an isolated nucleic acid encoding a ketolytic enzyme,
detecting the level of insulin resistance in the transgenic non-human mammal after administration of the compound, wherein a reduction in the level of insulin resistance identifies the compound as a candidate for the treatment of diabetes.

85. (canceled)
